(19) United States
(12) Patent Application Publication
(10) Pub. No.: US 2011/0104128 A1
(43) Pub. Date: May 5, 2011

(54) Device and Method for Transfecting Cells for Therapeutic Use

(75) Inventors: Laurence J.N. Cooper, Houston, TX (US); Dean A. Lee, Pearland, TX (US); Lisa Biswal, Houston, TX (US); Robert Raphael, Houston, TX (US); Thomas C. Killian, Houston, TX (US)

(73) Assignees: The Board of Regents, The University of Texas System; Office of the Board of Regents, Austin, TX (US)

(21) Appl. No.: 12/610,133

(22) Filed: Oct. 30, 2009

(51) Int. Cl. A61K 45/00; C12M 3/00; A61P 25/28; A61P 7/06

(52) U.S. Cl. 424/93.21; 435/285.2

(57) ABSTRACT

This invention generally relates to devices and methods for transfection of living cells using electroporation, in particular high throughput microfluidic electroporation, and to therapeutic uses of the transfected cells.

### STATEMENT AS TO FEDERALLY SPONSORED RESEARCH

This invention was made in part with Government funding and the Government therefore has certain rights in the invention.

### FIELD

This invention relates to molecular biology, physics, biophysics, microfabrication, microfluidics, genetic material therapy and medicine. In particular, it relates to devices and methods for stable and transient insertion of therapeutic nucleic acids into mammalian cells by electroporation and use of the transfected cells in the treatment diseases.

### BACKGROUND

There is a current trend to produce micro and nano scale devices that can perform physical, chemical, and biological processes on a small scale with the same efficiency as conventional macroscopic systems. These micro total analytical systems (μTAS) provide sample handling, separation, and detection on a single device using miniscule sample and reagent volumes. In fact, a variety of micro components such as pumps, valves, mixers, filters, heat exchangers, and sensors have been developed and used to create “lab-on-a-chip” devices.

Another current trend in the medical field has been development of cell-based therapy for the treatment of diseases. In its most basic manifestation, cell-based therapy involves the alteration of the genome of living cells whereby faulty genes that either do not express an essential protein or express a mutant protein, which may be non-functional or may function abnormally to produce a particular disease, are “repaired.” Since the genome itself is affected, the repaired gene will be passed on to daughter cells. The as of yet unfulfilled goal of gene therapy is the treatment of genetic diseases such as in-born errors of metabolism (e.g. Gaucher, Krabbe), hemophilia, cystic fibrosis, Down syndrome, Huntington's disease, dwarfism, sickle cell anemia, Tay-Sachs disease, phenylketonuria, amyotrophic lateral sclerosis (ALS, Lou Gehrig's disease), Parkinson's disease and many others. This type of cell-based therapy is formally termed “gene therapy,” because it is so defined by the FDA: “ . . . a medical intervention based on modification of genetic material of living cells. Cells may be modified ex vivo for subsequent administration or may be altered in vivo by gene therapy products given directly to the subject.”

An alternative to gene therapy is transient transfection of nucleic acids coding for desired proteins into cells where the proteins are either expressed on the cells' surface to direct or redirect the cells responsiveness to outside influences or are secreted by the cells to provide therapeutic biologics. The same diseases amenable to gene therapy with integrating vectors may be treated using cells that are transiently transfected.

While there is considerable cross-over among the techniques for effecting gene therapy and transient transfection, the most prevalent procedure for the former is by means of integrating vectors such as DNA plasmids, viruses, retroviruses, adenoviruses, adeno-associated viruses and the like. While viral gene transfection is extremely efficient, it is not without significant problems such as toxicity and other undesired side effects, difficulty in assuring the virus infects the correct target cell, ensuring that the inserted gene does not disrupt any other genes, etc. Transient transfection, since it does not involve interaction with the genome, circumvents many of the problems.

Transient transfection may be accomplished by a variety of mechanical, chemical and electrical means. Mechanical means of transfection include direct microinjection, particle bombardment with nucleic acid coupled to gold microparticles, sonoporation, and pressured infusion. Chemical transfection involves the use of agents capable of disrupting the plasma membrane sufficiently to permit exogenous materials such as therapeutic agents to cross. Chemical transfection agents include DEAE dextran, calcium phosphate, polyethylenimine and lipids. A fundamental problem with chemical transfection is toxicity; it has been posited that there is no chemical agent that doesn't have some toxic effect on cells.

Electrical techniques for transfection are dominated by electroporation, which involves application of a high-voltage electrical current to the cells, which causes disruption of the phospholipid bilayer of the plasma membrane resulting in the formation of pores in the membrane through which extracellular materials can pass. Since the electric potential across cell membrane rises about 0.5 to 1.0 volt concurrently with the formation of pores, charged molecules such as DNA are driven through the pores in a manner similar to electrophoresis. After removal of the electric field the membrane reseals leaving the cells intact. Electroporation can be accomplished by batch-processing cells in cuvettes or on multiwell plates and, more recently, using microfluidics. None of these methods as currently practiced is particularly amenable to mass production of transfected cells in clinically useful quantities except through propagation of the transfected cells to prepare the required number of cells.

Thus, as currently practiced, all cell transfection techniques, those mentioned above as well as the many others known to those skilled in the art are extremely labor intensive, inefficient, and typically rely on access to full-scale good manufacturing practice (GMP) facilities, biological safety level 2 (BSL2) at least, which renders them prohibitively expensive.

What is needed is an efficient, economic means of transfecting cells in therapeutically useful quantities and subsequently administering those cells to patients in need thereof, all in a clinical setting.

### SUMMARY

Thus in one aspect the present invention relates to a device for high throughput transfection of living cells, comprising:
optionally, a cell selection component capable of being operatively coupled to a source of living cells;
optionally, a cell focusing component:
   capable of being operatively coupled to a source of living cells if the cell selection device is not opted for or
   operatively coupled to the cell selection component;
optionally, a cell activation component:
   capable of being operatively coupled to a source of living cells if both the cell selection and cell focusing components are not opted for or, if the cell selection component is not opted for but the cell focusing component is,
   operatively coupled to the cell focusing component or if the cell focusing component is not opted for and the cell selection component is, operatively coupled to the cell selection component;
a high throughput electroporation component:
   capable of being operatively coupled to a source of living cells or
   if opted for, operatively coupled to the cell activation component or
   if the cell activation component is not opted for and the cell focusing component is, operatively coupled to the cell focusing component or
   if both the cell activation component and the cell focusing component are not opted for and the cell selection component is, operatively coupled to the cell selection component;
a source of DNA and/or RNA operatively coupled to the high throughput electroporation component;
optionally, a transfection detector component operatively coupled to the distal end of the high throughput electroporation component; and,
optionally, a cell separation component operatively coupled to the transfection detector.

In an aspect of this invention, the cell selection component comprises an apheresis component.

In an aspect of this invention, the cell focusing component comprises channels for funneling cells through the electroporation device one cell at a time.

In an aspect of this invention, the cell activation component comprises a chamber having an inlet operatively coupled to a source of activating substance, the chamber also being operatively coupled to the cell selection component, if opted for, the cell focusing component if the cell selection component is not opted for or capable of being coupled to a source of living cells if neither the cell selection nor the cell focusing components are opted for, and an outlet operatively coupled to the electroporation component.

In an aspect of this invention, the high throughput electroporation component comprises a plurality of microfluidic electroporation units, each unit comprising:
a first non-conductive support element, the element having a length with a proximal end and a distal end, a width and a surface;
a first conductive layer disposed over the surface of the first non-conductive support element;
a second non-conductive support element having a length and width substantially the same as the first non-conductive support element and a surface, the second non-conductive support element being substantially parallel to the first non-conductive support element with the surface of the second non-conductive support element facing the surface of the first non-conductive support element;
a second conductive layer disposed over the surface of the second non-conductive support element; wherein:
   the first conductive layer is no more than about 100 μm distant from the second conductive layer, the distance being maintained by a plurality of non-conductive spacers; wherein:
      the spacers extend from the proximal to the distal ends of the conductive surfaces thereby forming a plurality of channels extending substantially the full length of the conductive surfaces;
a pulse generator in electrical contact with the first conductive layer and the second conductive layer; and,
a positive displacement pump operatively coupled to a proximal end of the plurality of electroporation units; or,
a vacuum pump operatively coupled to a distal end of the plurality of electroporation units.

In an aspect of this invention, the transfection detector component comprises a fluorescence detector.

In an aspect of this invention, the cell separation component comprises channels that separate transfected cells from live-but-not-transfected cells and/for from dead cells.

In an aspect of this invention all the components are contained in a sealed housing having one or more inlets and one or more outlets for contact with the external environment.

In an aspect of this invention, all the components and the housing are sized to be implantable in the body of a patient.

An aspect of this invention is a method of treating a disease, comprising:
identifying a patient afflicted with a disease that is known to be, becomes known to be or is suspected of being responsive to treatment using transfected cells;
providing a source of living cells;
optionally selecting one or more cell types from the living cells;
optionally focusing the source of living cells or the selected cell types;
optionally activating the living cells or the selected cell types;
mixing the living cells or selected cell types with DNA and/or RNA;
electroporating the living cells or selected cell types in the presence of the DNA and/or RNA to give transfected living cells or selected cell types;
optionally detecting cells that have been transfected;
optionally separating transfected cells from living-but-not-transfected cells and/or from dead cells;
administering the transfected cells to the subject, wherein the transfected cells express a biotherapeutic agent; and,
repeating the above steps until treatment of the patient is complete.

In an aspect of this invention, in the above method the living cells or selected cell types are mixed with RNA.

In an aspect of this invention, in the above method at no point are the living cells or selected cell types propagated prior to administering them to the patient.

In an aspect of this invention, in the above method providing a source of living cells comprises:
providing a sterile container comprising one or more selected cell types;
providing a bodily fluid comprising living cells that has been previously collected from a subject and stored in a sterile container; and,
providing a subject from whom a bodily fluid containing living cells is taken and directly transferred under sterile conditions to the cell selection component,
if opted for, the cell activation component, if the cell selection component is not opted for, a cell focusing component, if the cell selection and cell activation components are not opted for or the electroporation component, if the cell selection, cell activation and cell focusing components are not opted for.

In an aspect of this invention, in the above method, the method is performed recursively.

In an aspect of this invention, in the above method performing the method recursively comprises step-wise providing a source of living cells by providing a patient in need of treatment, collection of a bodily fluid from the patient, subjecting the cells to the method of claim 8 and delivering transfected cells back into the patient and repeating the process as necessary, all under sterile conditions.

In an aspect of this invention, in the above method performing the method recursively comprises using Nucleofector® to electroporate the cells.

In an aspect of this invention, in the above method performing the method recursively comprises continuously collecting the bodily fluid from the patient, continuously subjecting the bodily fluid to the method of claim 8 and continuously delivering the transfected cells back into the patient in a closed, sterile cycle.

In an aspect of this invention, in the above method transfection is transient.

In an aspect of this invention, in the above method performing the method recursively comprises using the plurality of high throughput microfluidic electroporation units of this invention.

In an aspect of this invention, in the above method taking a bodily fluid from a patient comprises venipuncture, aphersis, an in-dwelling central catheter, a central intravenous catheter or a combination thereof.

In an aspect of this invention, in the above method the bodily fluid is blood or a component of blood.

In an aspect of this invention, in the above method selecting one or more cell types comprises apheresis.

In an aspect of this invention, in the above method the one or more selected cell types are selected from the group consisting of T cells, NK cells, B cells, dendritic (antigen presenting) cells, monocytes, reticulocytes, stem cells, tumor cells umbilical cord blood-derived cells, peripheral-blood derived cells and combinations thereof.

In an aspect of this invention, in the above method the stems cells are selected from the group consisting of hematopoietic stem cells and mesenchymal stem cells.

In an aspect of this invention, in the above method the one or more selected cell types are selected from the group comprising T cells, NK cells or a combination thereof.

In an aspect of this invention, in the above method activating the T cells and/or NK cells comprises contacting the cells with a cytokine or a growth factor.

In an aspect of this invention, in the above method the cytokine is IL-2.

In an aspect of this invention, in the above method RNA is selected from the group consisting of mRNA, microRNA and siRNA.

In an aspect of this invention, in the above method the RNA and/or DNA code for a biotherapeutic agent.

In an aspect of this invention, in the above method the biotherapeutic agent is selected from the group consisting of a chimeric antigen receptor, an enzyme, a hormone, an antibody, a clotting factor, a Notch ligand, a recombinant antigen for vaccine, a cytokine, a cytokine receptor, a chemokine, a chemokine receptor, an imaging transgene, a co-stimulatory molecule, a T-cell receptor, FoxP3, a luminescent probe, a fluorescent probe, a reporter probe for positron emission tomography, a KIR deactivator, hemoglobin, an Fc receptor, CD24, BTLA, a transposase, a transposon for Sleeping Beauty, piggyBac and combinations thereof.

In an aspect of this invention, in the above method the patient is a mammal.

In an aspect of this invention, in the above method the mammal is a human being.

In an aspect of this invention, in the above method the human being is a pediatric patient.

In an aspect of this invention, in the above method the disease is selected from the group consisting of a pathogenic disorder, cancer, enzyme deficiency, in-born error of metabolism, infection, auto-immune disease, cardiovascular disease, neurological disease, neuromuscular disease, blood disorder, clotting disorder and a cosmetic defect.

### BRIEF DESCRIPTION OF THE DRAWINGS

FIG. 1 shows a DNA plasmid vector which serves as in vitro template for translation to generate mRNA.

FIG. 2 shows formaldehyde-agarose gel electroporation of in vitro transcribed CD19R and CD19RCD28 mRNAs. These mRNAs code for a chimeric antigen receptor with specificity for CD19.

FIG. 3A shows a FACS analysis of Jurkat cells (T cell), NK92 cells (NK cells) electroporated with CD19R and CD19RCD28 mRNAs synthesized from the vectors. Cells were analyzed with 2D3 Alexa-labeled CD19R-specific mAb (made at MDACC) and NK-cell marker CD56. Propidium iodide (PI) staining was used to determine the viability of the cells after electroporation.

FIG. 3B shows the determination of the fate of mRNA in cells after electroporation as determined by Cy5-labeled CD19R mRNA as wells as 2D3 Alexa-labeled CD19R-specific antibody.

FIG. 4 is FACS analysis of OKT-3/IL-2 activated T-cells and Jurkat cells electroporated with CD19RCD28 mRNAs synthesized from the T7 based CD19RCD28 plasmid vectors.

FIG. 5 is schematic illustration of an embodiment of the present invention for creating a focused stream of single cells using microfluidics.

FIG. 6 shows a side view of cell traveling through multiple electric fields to improve transfection efficiency.

FIG. 7 shows detection of cell transfection by an embodiment of the present invention using fluorescence.

FIG. 8 shows summary of a clinical trial design for an embodiment of the non-integrating method described herein.

FIG. 9 shows a schematic representation of biodistribution of infused therapeutic agents as derived by NIP technology.

FIG. 10 shows phenotype and function of genetically modified T cells.

FIG. 11 shows binding of anti-CD20-IL-2 ICK to B cells and T cells.

FIG. 12 shows effect of immunocytokine (ICK) on persistence of adoptively transferred T cells.

FIG. 13 shows combined anti-tumor efficacy of ICK and CD19-specific T cells.

FIG. 14 shows measurement of both T-cell persistence and anti-tumor effect of immunotherapies in individual mice.

FIG. 15 shows a microfluidic electroporation unit of this invention.

FIG. 16 shows one embodiment of a “GMP-in-a-box” of this invention wherein the microfluidic electroporation unit of FIG. 15 is encased in a housing that can comprise a disposable cartridge.

FIG. 17 shows a number of the microfluidic electroporation units of FIG. 15 arrayed in housing such that the device and method hereof is capable of high throughput operation.

FIG. 18 shows a microfluidic electroporation unit of this invention sized down to be implantable in the body of a patient.

### DETAILED DESCRIPTION

As used herein, “high throughput” refers to the production of a sufficient number of transfected cells to be therapeutically effective in a clinically relevant time-frame. To be therapeutically effective the transfected cells must produce a selected biotherapeutic agent in sufficient quantity to have a beneficial effect on the health and well-being of a patient being treated. A beneficial effect on the health and well-being of a patient includes, but is not limited to: (1) curing the disease; (2) slowing the progress of the disease; (3) causing the disease to retrogress; or, (4) alleviating one or more symptoms of the disease. As used herein, a biotherapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, “microfluidic” retains the meaning that would be understood by those skilled in the art; that is, in general it refers to a device that has one or more channels with at least one dimension less than 1 mm. The devices of the current invention have a dimension, the distance between the two substantially parallel conductive surfaces that is no more than about 100 μm, preferably no more than abut 50 μm and thus qualified as microfluidic.

As used herein, “electroporation,” “electroporating” and other versions of the word likewise have the meaning generally ascribed to them by those skilled in the art. That is, in brief, electroporation refer to the process of subjecting a living cell to an electric field such that, when the voltage across the plasma membrane of the cell exceeds its dielectric strength, the membrane is disrupted and pores form in it through which substances, in particular polar substances that normally are unable to traverse the membrane, can pass and enter the cytoplasm of the cell. If the strength of the electric field coupled with the time of exposure is properly selected, the pores reseal after the cell is removed from the electric field.

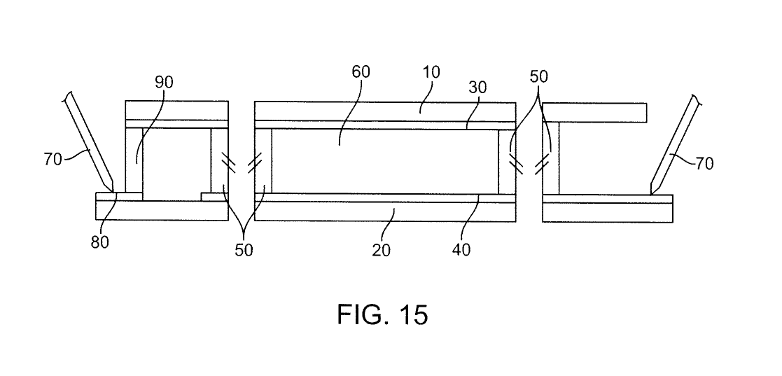

As used herein, an “electroporation unit” refers to all of the elements of a device necessary to effect the high throughput electroporation of living cells. A diagram of an exemplary but non-limiting electroporation unit of the current invention is shown in FIG. 15. In FIG. 15, the view is looking down a channel of the device from a proximal end of the device to a distal end of the device. Only a single channel is shown whereas the device may comprise a large number of parallel channels. In FIG. 15, non-conductive support elements 10 and 20 are made of any type of material having sufficient mechanical strength to maintain the mechanical integrity of the unit. They may be made of such material as a glass including without limitation Pyrex®, a ceramic, a non-conductive polymer, a mineral such as sapphire. It is presently preferred that the support elements be made of a biocompatible substance, that is a substance that will not have a deleterious effect on cells and other biological substances that might come in contact with the element. Support elements 10 and 20 are coated with conductive layers 30 and 40. Conductive layers 30 and 40 can be made of any conductive biocompatible material such as, without limitation, a biocompatible conductive metal such as, without limitation, gold, or a biocompatible conductive polymer. They may be applied to the surfaces of the support elements by any means known or as becomes known in the art for accomplishing such including, without limitation, microlithography, vapor deposition, plasma deposition, and the like. If the conductive layer material does not adhere to the surface of the support elements, a primer layer to which the conductive material will adhere may be first applied to the support surfaces. The distance between the conductive surfaces is maintained by a plurality of non-conductive spacers 50 that extend essentially the full length of the conductive layers and are contiguous with the layers so as to form a number of discrete channels 60 in the unit. The non-conductive spacers, like the non-conductive support elements, can be made of any non-conductive material capable of maintaining the mechanical integrity of the structure such as, without limitation, a non-conductive polymer. The distance between the conductive surfaces as established by the spacers is not greater than about 100 μm, preferably at present not more than 50 μm. The distance between spacers can be any that is desired. Finally, the electroporation unit comprises a pulse generator that is in electrical contact with the conductive surfaces, one lead of the generator being in contact with each of the conductive surfaces. As depicted in FIG. 15. electrical contact is made using Pogo® pins 70, which are well known by those skilled in the microelectronics art. The right hand pin is in contact with conductive layer 40 while the left hand pin is in contact with conductive material 80, which may be the same as or different than conductive layers 30 and 40 and conductive material 80 is in electrical contact with conductive vertical element 90 that, in turn, is in electrical contact with the conductive layer 30. The ends of the pins that are not shown in contact with the device are of, course, connected to the pulse generator.

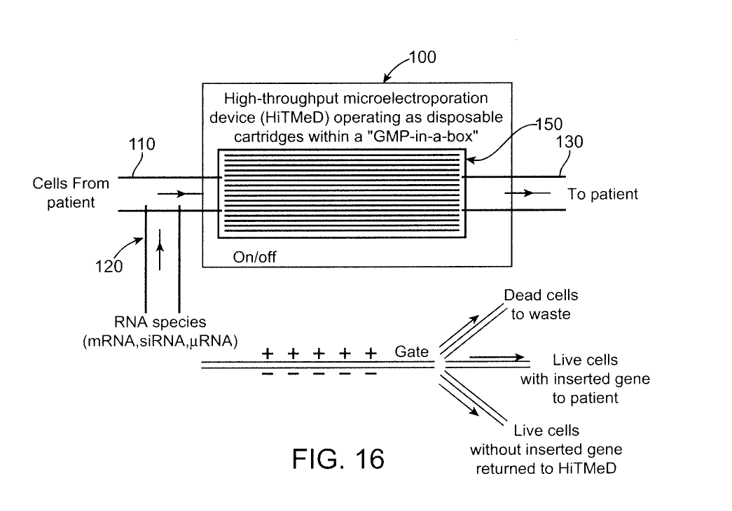

The microfluidic electroporation units (MEU) described above may be used individually as illustrated in FIG. 16. In FIG. 16 MEU 105 is contained in a sealable sterile housing 100, which may be reusable or a disposable cartridge. The patient is the source of cells to be transformed as is shown in FIG. 16, the inlet 110 labeled “cells from patient.” The cells are collected from the patient by tapping a selected source of bodily fluid such as, without limitation, venipuncture of a vein from which blood is drawn. Other sources include an indwelling catheter or a central intravenous catheter. Being mixed with the cells from the patient prior to their entry into the MEU is a stream of an RNA species from inlet 120 with which the cells will be transformed. Inlet 120 is shown in FIG. 16 as being outside the housing or cartridge; however, it may be connected to the housing itself such that the cells and the RNA mix inside the housing just prior to electroporation. Once the cells have been electroporated and the RNA has entered the cells, the transformed cells exit the MEU and the housing through outlet 130 and are returned to the patient through the same of a different route, i.e., the same venipuncture that was used to collect the cells in the first place or they may be returned by means of a separate venipuncture. If desired, transformed cells can be separated from living-but-not-transformed and from dead cells as shown in the second diagram of FIG. 16. The cell separation component may be external to housing 100 or it may be internal so as to render the entire apparatus as self-contained as possible.

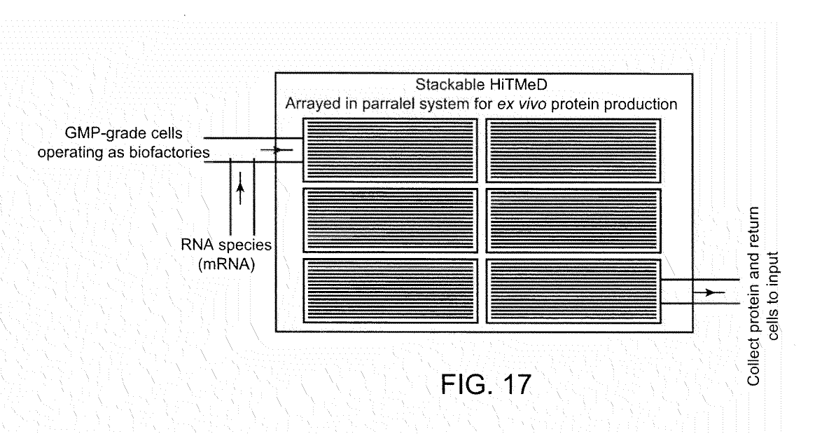

While MEUs may be used individually as shown in FIG. 16, preferably at present they may be used in arrays of multiple MEUs to facilitate high throughput transfection of cells an enhance the therapeutic utility of the devices and methods of this invention. A non-limiting schematic of stacked MEUs units is shown in FIG. 17.

The overall size of a device of this invention will depend on the size of the various components and the housing containing some or all of them. In one aspect of this invention, the components may be of any acceptable size because it is envisioned that the method herein will be used to transform cells from a fluid taken from the body of a patient in a separate step, the fluid being collected from the patient under sterile conditions, e.g., without limitations, standard blood banking practices, and then separately introduced into a device of this invention while maintaining sterile conditions throughout. The device may be physically situated at the site of fluid collection such as, without limitation, a hospital room or an out-patient clinical setting, or the fluid may be collected at one location and then transported to another location, for example without limitation, another room or a facility in another state or country, where the device is located. The transfected cells, still under strict sterile conditions, can then be transported back to where the patient is located for re-introduction into the patient's body.

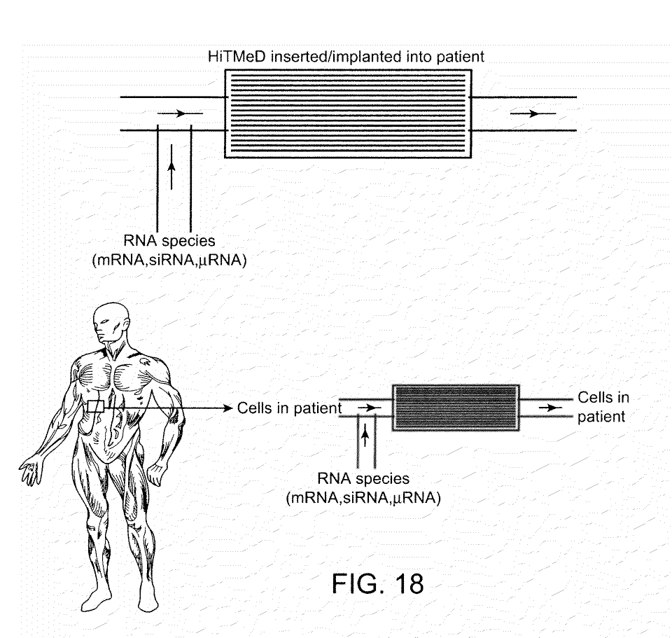

It is, however, envisioned that the components and the housing can be sized so as to be implantable in the body of a patient as shown in FIG. 18. Micro scale versions of many of the components of the devices herein, other than the novel MEUs of this invention, are either available or will be achievable by those skilled in the art based on the disclosures herein.

As used herein, “genetic material” refers to DNA or RNA that, when inserted into a living cell, expresses or leads to the expression of a desired protein regardless of whether the genetic material is actually integrated into the organism's genome or simply inserts into the nucleus and/or cytoplasm and makes use of the replicatory machinery therein to express the protein.

In particular at present, “genetic material” refers to RNA (such as micro-RNA and small inhibitory RNA) that, when inserted into a living cell, alters expression of a desired protein regardless of whether the genetic material is actually integrated into the organism's genome or simply inserts into the nucleus and/or cytoplasm.

In one aspect the present invention relates to a microfluidic electroporation device and method of use for efficient, reproducible, sporadic or continuous insertion of genetic material, fluorochromes (tags) and/or proteins into cells by electroporation. For example, without limitation, an integrated system that is capable of high throughput electroporation of a large number of clinical grade cells in parallel fashion is an aspect of this invent. The process may be carried out is numerous ways including, without limitation, using individual component devices with manual transfer of the product of one component into the next component to rendering the entire process, from obtaining the desired cell type for transfection to the delivering the transfected cells to a subject in need thereof, in a totally closed system. Further, it is contemplated that all of the components may be miniaturized such that the entire closed system can be implanted in the body of the subject for continuous long-term therapy. The closed systems, whether macro or micro scale, can mimic the operating condition provided by a GMP facility or one that operates under standard blood banking protocols. Thus, what the devices of the current invention in effect offer is a self-contained “GMP-in-a-box” that will facilitate the transfer of integrating and non-integrating gene and other nucleic acids into cells under standard blood-banking and good manufacturing practices as established by the FDA and AABB (American Association of Blood Banks). That is, cells can be recursively collected from a subject, for example without limitation, by venipuncture or apheresis, a nucleic acid coding for a desired protein can be transferred into the cells or into a desired subset of cells such as, without limitation, T and NK cells and the modified cells can be re-infused into the patient to effect treatment, all in a sterile closed system that can be operated in a clinical setting. Advantages of this process compared to those currently in use in gene therapy and non-integrating cell therapy include, without limitation, the adoptive transfer of minimally manipulated cells at a cost substantially below that of ex vivo culturing and an inherent improvement in the biologic functioning of the modified cells since cell differentiation, which accompanies propagation needed to achieve clinically-meaningful numbers of cells is not required. That is, the devices of the current invention can be coupled with high throughput so as to allow patients receiving gene transfer therapy to receive back large numbers of cells within hours of collection followed by gene transfer. This constitutes a fundamental shift in the way gene therapy is perceived.

In sum, until now, the introduction and expression of genes has required major investment in research, development, manufacturing and regulatory support. While this has resulted in the development of state-of-the-art GMP facilities that are capable of executing complex manufacturing and release processes, the technology is expensive and time consuming. Due primarily to the expense involved and concerns over genotoxicity from integrating vectors, just a few patients around the world are currently or ever will be able to benefit from integrating gene therapy. The ability to operate the current invention, especially using non-integrating cell transfection therapy, in a clinical setting means that recombinant therapeutic proteins (such as produced by gene therapy) will be available to a many more patients of diverse economic means than is even imaginable using current technologies including, significantly, patients in under-developed and developing nations. The operation of the current technology brings the GMP process closer to the bedside to deliver recombinant therapeutic proteins in situ (in vivo) which will greatly broaden the number of patients who can benefit.

The devices and methods described herein will be amenable to a variety of applications, e.g., gene therapy for the prevention and cure of inheritable diseases and both gene therapy and transient transfection treatment of diseases known to be, or become known to be or that are suspected of being susceptible to treatment by such cell-based therapy. A particularly notable disease for which transient transfection may be useful is cancer and replacement therapy for in-born errors (e.g., Gaucher and hemophilia).

A device of the present invention can not only introduce desired genetic material into cells but also can monitor the cell's responses. This can be accomplished by providing a marker that will be co-expressed along with the desired genetic material by transfected cells and which can be detected by various means to identify cells that in fact have been transfected. While numerous such marker techniques are known to those skilled in the art and all are within the contemplation of this invention, one non-limiting example of such is use of a fluorescent tag that can be detected by a fluorescence detector and positron emission tomography and single photon emission computed tomography.

The efficiency of the device and method of the present invention lend itself readily to adoptive transfer of minimally manipulated cells with reduction in costs associated with extensive ex vivo culturing as well as improvements in the biologic functioning of the genetically modified cells since cell differentiation, which accompanies propagation needed to achieve clinically-meaningful numbers of T and NK cells, can be avoided.

The present invention can improve the efficiency of the transfer of genetic material into immune-derived cells for the treatment of cancer using novel cell electroporation and gene material delivery techniques.

Non-viral gene transfer has been used to introduce DNA plasmids and RNA species expressing desired transgenes. Currently, non-viral gene transfer uses commercially available technology to achieve ex vivo electrotransfer of RNA and DNA in cells in cuvettes. This method of gene transfer, however, is inefficient due to low transfection and integration efficiency and is not readily amenable to GMP processes due to difficulties in engineering a closed and/or sterile system to accomplish the transfer.

To address the above problem, the present invention provides microfluidic genetic material transfer devices which can be operated within most blood banking centers in developed and developing nations, thereby significantly broadening the distribution of technology analogous to gene therapy. These devices can be coupled with high throughput so as to allow patients receiving genetic material therapy to reiteratively receive back large numbers of cells within hours of collection and modification using the method of this invention, resulting in a fundamental shift in the way such therapy is perceived and delivered.

An aspect of this present invention is a multi-stream channel comprising parallel lanes. The multi-stream channels can allow cells and buffer solutions to flow through while maintaining their respective streamlines due to low Reynolds numbers for the respective streams resulting in laminar flow. The multi-stream channel can further include a plurality of electrodes in a pattern that generates multiple electroporation zones in the channel. The electroporation zones can include mechanisms to control the duration and electric voltage of electroporation so as to control the number and size of pores on a cell flowing through the channel. The size of pores can range from about 2 nm to about 10 nm. An array of multistream channels are also within the contemplation of this invention to provide a high throughput device capable of producing therapeutically significant quantities of transfected cells in a relatively short period of time.

In an aspect of this invention, a method of genetic material therapy is provided that comprises: identifying a patient suffering from a disease; selecting a cell-type for treatment of the disease; removing a fluid containing cells of the selected cell-type from the patient's body; separating the cells from other constituents of the fluid; optionally activating the separated cells; electroporating the separated cells; contacting the electroporated cells with one or more therapeutic DNAs and/or RNAs to form non-integrated DNA- and/or RNA-containing cells; optionally evaluating the DNA- or RNA-containing cells for conformance with release criteria; returning the DNA- and/or RNA-containing cells into the patient's body; and, repeating the removing, separating, optionally activating, electroporating, contacting, optionally evaluating and returning as necessary to treat the disease.

As used herein, a “source of living cells” refers to any source known to those skilled in the art. Examples include, but are not limited to, commercial sources of specific cell types or mixtures thereof, whole blood either taken from a subject and transferred to a storage container for later use in the methods herein or taken from a subject and transferred directly to a device of this invention.

If a source, such as whole blood, that contains a mixture of many cell types is used it may be desirable to separate out the cells of interest using a “cell selection component.” If cell selection is opted for, any means known to those skilled in the art may be employed. These include, without limitation, centrifugation techniques, i.e., density-based techniques such as apheresis, magnetic techniques employing antibodies to tag specific cell types with small magnetic particles that are later isolated and use of antibody complexes to remove unwanted cells from the selected cell type, etc.

The cell-type can be any type of cell known or found to be useful for a particular therapeutic purpose. That is, cells such as, without limitation, T cells, NK cells, dendritic cells (or antigen presenting cells), B cells, monocytes, reticulocytes, fibroblasts, hematopoietic stem/progenitor cells, mesenchymal stem cells, other stem cells, tumor cells, umbilical cord blood-derived cells and peripheral-blood derived cells may be used.

The cell-type can be numerically expanded and/or cultured ex vivo prior to insertion of the nucleic acid.

The DNA and/or RNA can code for therapeutic agents including, without limitation, an enzyme, a chimeric antigen receptor, a hormone, an antibody, a clotting factor, a notch ligand, a recombinant antigen for vaccine, a cytokine, a cytokine receptor, a co-stimulatory molecule, a T-cell receptor, FoxP3, a chemokine, a chemokine receptor, a luminescent probe, a fluorescent probe, a reporter probe for positron emission tomography, a KIR deactivator, hemoglobin, Fc receptors, CD24, BTLA, somatostatin, a transposase, a transposon for Sleeping Beauty or piggyBac and combinations of any of the foregoing. The RNA can be chemically modified to improve persistence. Further, the RNA can be prepared in vitro from DNA plasmid which has been modified (e.g. a polyA tail can be added and/or untranslated region from beta-globin can be included) to confer improved persistence of the RNA species (Holtkamp et al., Blood, (2006) 108:4009-17). The RNA can be any of mRNA, siRNA and microRNA or combinations thereof. If desired, the RNA species can be combined with DNA species, such as the electrotransfer of mRNA transposase from, for example without limitation, Sleepy Beauty (Wilber et al., Mol. Ther. (2006) 13:625-30) or piggyBac (Wilson et al., Mol. Ther. (2007) 15:139-45.)) and a DNA plasmid transposon such as that coding for, without limitation, a chimeric antigen receptor.

The above procedures can be carried out in a variety of ways. Preferably at present, all steps are performed in a closed recirculating system; that is providing a source of living cells, optionally selecting certain cells from the source, optionally focusing the selected cells, optionally activating the selected cells, mixing the cells with DNA and/or RNA, electroporating the cells, optionally detecting transfected cells and then collecting the transfected cells is accomplished in a closed sterile unbreached system. For example, providing a source of of living cells can be accomplished by, without limitation, venipuncture, apheresis, use of an in-dwelling central catheter or use of a central intravenous catheter. Selecting one or more selected cell types can also comprise, without limitation, apheresis. Cells may also be obtained by biopsy or surgery. Activating the cells can be accomplished by treating the cells with a substance that is causes the cell to undertake a particular function. For example without limitation, T and NK cells are known to become cytotoxic when activated by exposure to cytokines, such as IL-2, or growth factors. Electroporating cells can comprise using Nucleofector®. Contacting the electroporated cells with one or more therapeutic DNA(s) and/or RNA(s) can comprise contacting the cells with a fluid containing the therapeutic DNA(s) and/or RNA(s). Electroporation and contacting the electroporated cells with a fluid containing the therapeutic DNA(s) and/or RNA(s) can be performed substantially simultaneously. That is, the cells can be mixed with the DNA and/or RNA prior to subjecting the cells to electroporation. Returning the therapeutic DNA- and/or RNA-containing cells can comprise the same route by which the cells were provided in the first place, i.e., venipuncture, an in-dwelling central catheter, a central intravenous catheter, etc., or it may be accomplished using a canulating lymphatic system.

Any disease known to be, or that may become known to be in the future, or that is suspected of being, amenable to gene therapy can be treated using the methods and devices of this invention. Cancer, for instance, is presently known to be such a disease. Thus, a genetic material transfer therapy for cancer using the methods and devices of this invention might comprise removing a fluid containing T-cells and/or NK cells by apheresis, separating the T-cells and/or NK cells using a microfluidic cell separator, activating the cells by contacting them with IL-2, and then electroporating them using Nucleofector®. Contacting the electroporated T-cells and/or NK cells with therapeutic DNA and/or RNA can comprise contacting them with mRNA coding for a CD19-specific chimeric antigen receptor. Electroporation and contact with the mRNA coding for CD19-specific chimeric antigen receptor can be conducted substantially simultaneously. The CD19-specific chimeric antigen receptor can comprise CD19RCd28.

As used herein, a “subject” refers to any living entity that might benefit from treatment using the devices and methods herein. As used herein “subject” and “patient” are used interchangeably. A subject or patient refers in particular to a mammal such as, without limitation, cat, dog, horse, cow, sheep, rabbit and preferably at present, a human being that may be an adult patient or a pediatric patient.

### Electroporation

A previously mentioned herein, electroporation is a well-established method for delivery of drugs and genes into cells. The basic concept of electroporation is that controlled application of an electric field to a mammalian cell membrane can temporarily increase membrane permeability as a result of the formation of nano-scale pores in the membrane. The use of microfluidic devices for cell electroporation is, however, novel and offers several advantages compared to current electroporation methods. For instance, microelectronic patterning techniques can reduce the distance between the electrodes in the microchips such that low voltages can be used to generate high electric field strengths. Cell handling and manipulation should also be easier since the channels and electrodes can be comparable in size to cells. Cell electroporation, separation and detection can be integrated on a single platform. Transformation efficiency can be improved. A micro-electroporation device may be integrated with other devices in a complex analyzer. Such advanced integration will be possible because cellular manipulations in the present invention are performed in simple flow systems.

As shown in Example 1, a chimeric antigen receptor (CAR) can be successfully introduced into cells by electroporation and thereafter expressed by the cells.

### Recirculating Closed System

As noted previously, an aspect of the present invention is a recirculating closed system for recursively extracting cells from a patient, electroporating them, transiently transfecting RNA or DNA into them and then returning them to the patient where expression of the transfected gene provides the desired therapeutic result. The recirculating closed system can include:
a fluid removal component having a proximal and a distal end and a lumen extending from the proximal to the distal end wherein the proximal end of the fluid removal component is inserted into a vein of the patient;
a first tube having a proximal and a distal end, the proximal end of which is coupled to the distal end of the fluid removal component;
a cell separation device having an inlet and an outlet wherein the distal end of the first tube is coupled to the inlet of the cell separation component;
a second tube having a proximal and a distal end, the proximal end of which is coupled to the outlet of the cell separation component;
an electroporation component having an inlet and an outlet wherein the distal end of the second tube is coupled to the inlet of the electophoresis component;
a third tube having a proximal and a distal end, the proximal end of which is coupled to the outlet of the electroporation component and the distal end of which is coupled to a proximal end of a fluid return component, a distal end of which is inserted into a vein of the patient.

In an aspect of this invention, the cell separation component and the electroporation component can be directly coupled to one another, that is, there is no second tube.

Likewise, the fluid removal component and the fluid return component can be one and the same. For example, without limitation, the fluid removal component and the fluid return component can comprise a single needle or an in-dwelling central intravenous catheter.

The recirculating closed system can comprise a single channel design that can electroporate single cells in a flow-through manner. An illustrative schematic of a channel design is shown in FIG. 5, where cells and buffer solutions flow in alternating lanes of a multi-stream channel. Because of the low Reynolds number, viscous forces predominate over inertial forces, laminar flow ensues and there is no pronounced convective mixing of the solutions. Thus, the fluids in each lane can maintain their respective streamlines and can be directed down the channel with mixing of solutes occurring only due to the relatively slow process of diffusion. Low Reynolds number flows can be used to focus a solution of cells into a single stream of cells.

Electrodes can be patterned into the channels using any suitable technique such as microlithography. Multiple electroporation zones can be created to control transfection efficiency. For example, a single cell may travel through multiple sets of electrodes before being transfected, thereby introducing multiple electroporation zones, as illustrated in FIG. 2, which can increase the probability of transfection and thus overall transfection efficiency, and one or a plurality of cross-channels can be used to introduce desired reagents, RNA, and/or media to the electroporated cells. Other factors include electric field strength for electroporating the cells and the rate of fluid flow which can be controlled so that cells are exposed to electric fields for a desired amount of time.

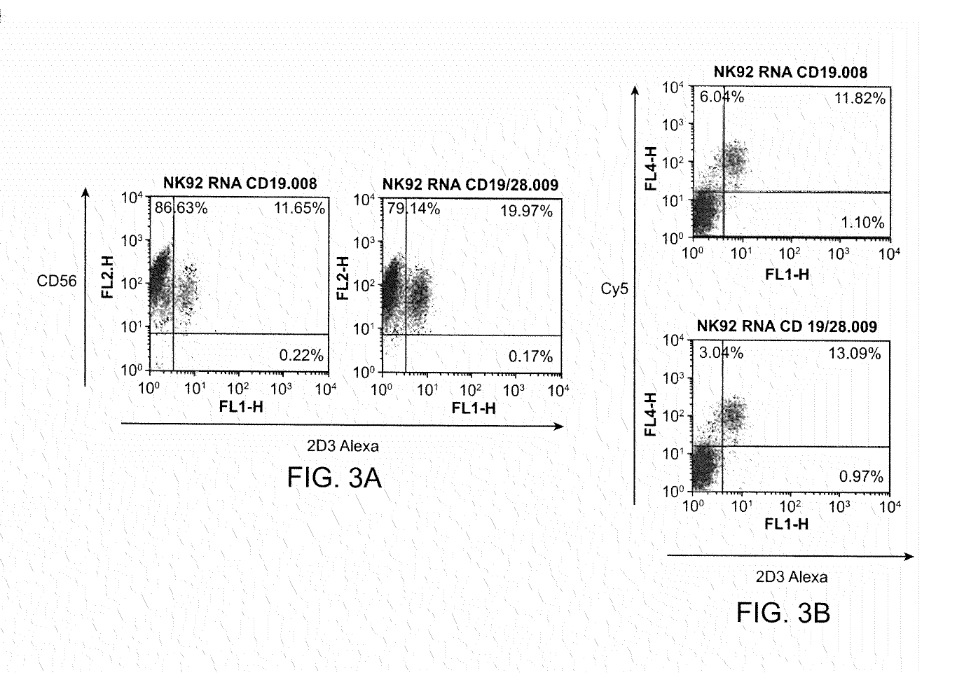

The recirculating closed system can incorporate a detection device to measure the efficiency of the system. For example, fluorescence labeling technology can be used to determine the efficiency of the system. Such a detection scheme can include an optical detection method that uses a membrane-impermeable fluorescent stain to monitor cellular membrane integrity (Yeh et al., J. Immunol. Methods (1981) 43:269-75, Schmidt et al. Cytometry (1992) 13:204-08). In addition, by transfecting electroporated cells with fluorescently-labeled target RNA and then measuring intracellular fluorescence not only how many cells were successfully electroporated but also how many tagged RNA molecules were transfected into the cells can be monitored. FIG. 3 shows a fluorescence labeling technology using cuvettes. This method permits evaluation of electrical parameters, voltage and pulse length needed for optimal cell membrane permeabilization. Further, whether compounds expected to stabilize membrane pores and thereby improve transfection efficiency are in fact doing so can be examined.

Microfluidic devices of this invention can be used to separate T and NK cells from other cells in the blood to avoid electroporation of the other cells. The T and NK cells can then be directed to channels which have an orifice plate which focuses the electric field and allows for single-cell electroporation with high efficiency. The electric field can be tailored by the orifice plate, allowing control of the magnitude and localization of the transmembrane voltage. Since electroporation of a cell results in a resistance change of the membrane, membrane permeation can be detected by characteristic ‘jumps’ in current that correspond to drops in cell resistance. The microfluidics device can, of course, be a high throughput device.

A plurality of channels can be created on a microfluidic device described herein according to the above procedures. For example, an array of channels can be created each of which can be used for single cell electroporation. Arrays of electrodes can likewise be created to perform multiple electroporation operations, which can last for hours, days or even months, preferably at present from about twelve to about twenty-four hours.

The microfluidic device can include disposable parts or components such as, for example, disposable microfluidic electrotransfer cassettes to avoid cross-contamination.

### Method of Use

The method and system described herein have a variety of applications. For example, the system and method can be used for recursive electrotransfer of DNA and/or RNA species, e.g., mRNA, to enforce gene expression, siRNA to down regulate disease causing gene expression and microRNA to regulate gene expression for integrating and non-integrating gene transfer. The transgenes can be used to express a protein or peptide in a cell or an organism using the method describe herein, which include, but are not limited to, genes expressing enzyme, e.g. glucocerebrosidase and galactocerebrosidase; clotting factors; chimeric antigen receptors (including humanized sequences); hormones, e.g., insulin; antibodies; clotting factors, e.g., hemophilia factors; Notch ligand; recombinant antigens for vaccines; cytokines; cytokine receptors; proteins or peptides expressed by imaging transgenes (e.g., thymidine kinase, iodine simporter, somatostatin receptor); co-stimulatory molecules; T-cell receptors; FoxP3; chemokines; chemokine receptors, e.g., CXCR4; luminescent probes; fluorescent probes; genes to de-activate KIR; hemoglobin; Fc Receptors; CD24; BTA; or somatostatin.

The transgenes can be expressed in human and non-human cells including, but not limited to: T cells; NK cells; B cells; monocytes; red blood cells (reticulocytes); stem cells, e.g., hematopoietic stem cells, mesenchyal stem cells; tumor cells; umbilical cord blood-derived cells; peripheral-blood derived cells; or cells that have undergone ex vivo numerical expansion.

### Method of Clinical Trials

The efficient introduction and expression of desired genes into viable immune cells such as T cells makes possible a new class of clinical trials based on the recursive infusion of genetically modified cells. This can have major advantages over current trial design as it (i) does not require integrating transgenes and can avoid the need for oversight by National Institutes of Health Office of Biotechnology Activities (NOH OBA) with associated stringent regulatory oversight and down-stream long term follow up expenses, (ii) avoids the need for production of expensive vectors (such as retrovirus or lentivirus) for transfection of immune cells, (iii) allows genetically modified cells to be available on demand, and (iv) uses a minimally-manipulated cell product which maintains in vivo viability (avoid replicative senescence associated with extensive ex vivo propagation) and avoids in-depth and expensive release testing.

For example, the microfluidic device described herein can be used to assess the efficacy of recursive adoptive transfer of autologous CD19-specific T cells in patients with chemo-refractory (lethal) B-lineage acute lymphoblastic leukemia. An inter-patient dose escalation can evaluate feasibility of giving 1 to 7 doses of 10⁹/m² CD19-specific T cells over a two-week period. Correlative studies can establish persistence of infused cells based on imaging technologies (e.g., PET imaging) and excretion of beta-HCG as well as determine the potential for an immune response against infused T cells.

The release/in-process testing for the infusion of CD19-specific genetically manipulated T cells are summarized in Table 1 below. These tests can be modified by an ordinary artisan to suit the application and gene expression desired as described in Table 2.

TABLE 1
Summary of “Release” assay and “In Process” testing

| Test | Release Criteria | In-Process Tests | Test Method |
|---|---|---|---|
| Sterility | Negative for | | Gram and KOH |
| | bacteria and fungi | | stains |
| | | Negative for bacteria | |
| Sterility | | at 14 days; Negative | U.S.P. |
| | | for fungi at 28 days | |
| Mycoplasma | | Negative for | PCR |
| assay | | mycoplasma | |
| Endotoxin | <5 EIU/Kg recipient | | |
| assay | body weight/hour of | | Chromogenic LAL |
| | T-cell infusion | | |
| Chimeric: ζ | | 75-kDa Protein Band | Western Blot with |
| receptor | | | human CD3ζ-specific |
| expression | | | primary Ab |
| Cell surface | ≧90% CD3⁺ and ≧10% | | Flow cytometric |
| phenotype | Transgene⁺ | | evaluation |
| Viability | ≧60% Viable | | Trypan blue |
| | | | exclusion test |
| CD19-specific | ≧30% Specific | | |
| cytolytic | lysis at 50:1 (E:T) | | 4 hr non-radioactive |
| activity | against a CD19⁺ | | lysis assay |
| (potency) | cell line | | |

TABLE 2
Summary of assays to assay expression of introduced genes

| Test | Criteria | Test Method |
|---|---|---|
| Sterility | Negative for | Gram and KOH |
| | bacteria and fungi | stains |
| Sterility | Negative for | U.S.P. |
| | bacteria at 14 days; | |
| | Negative for fungi | |
| | at 28 days | |
| Mycoplasma | Negative for | PCR |
| assay | mycoplasma | |
| Endotoxin | <5 EIU/Kg recipient | Chromogenic LAL |
| assay | body weight/hour of | |
| | T-cell infusion | |
| Introduced | Protein Band | Western Blot |
| gene | | |
| expression | | |
| within cells | | |
| Introduced | Protein expression | Flow cytometry |
| gene | | |
| expression on | | |
| cells | | |
| Introduced | Protein expression | ELISA or equivalent |
| gene | | |
| expression | | |
| outside of cells | | |
| Cell surface | ≧90% CD3⁺ | Flow cytometric |
| phenotype | | evaluation |
| Viability | ≧60% Viable | Trypan blue |
| | | exclusion test |

Non-integrative plasmid (NIP) technology, shown in Example 2, can be used to ensure that the genetically modified infused cells will (i) numerically proliferate and survive in vivo (ii) express the transgene appropriately and (iii) home to the disease site (e.g., tumor site). For example, two transgenes can be used to monitor the persistence/biodistribution of the genetically modified cells in vivo.

The transgene can be tagged with beta-HCG (human choriogonadotrophic hormone), the secretion of which can be used as a measure of gene transfer and beta-HCG excretion in urine can be used as a measure of in vivo survival of infused genetically modified cells. This information in turn will provide for a measurement of tumor killing vis-a-vis the persistence of the infused cells.

Immune-based therapies based on transient gene transfer to cells (e.g., to T and NK cells) have a variety of applications. The uses of non-viral gene transfer can be used to introduce RNA and DNA to deliver transgenes to achieve personalized medicine using cost-effective technology which can be broadly implemented.

The method described herein can be used as a therapeutic measure in the field of pediatric oncology. For example, a pediatric patient can undergo apheresis and reinfusion of genetically modified cells the same day using blood banking practices already in place. This can allow the development of investigator-initiated pediatric oncology drugs/therapeutics based on the patient's immune system leading to multi-institution gene therapy treatments recruiting large numbers of patients, leading to a portable genetic modification system at low cost and applicable to the application of genetically modified immune cells for multiple classes of neoplasms and pathogens.

### EXAMPLES

### Example 1

### Electroporation of mRNA to T-Cells

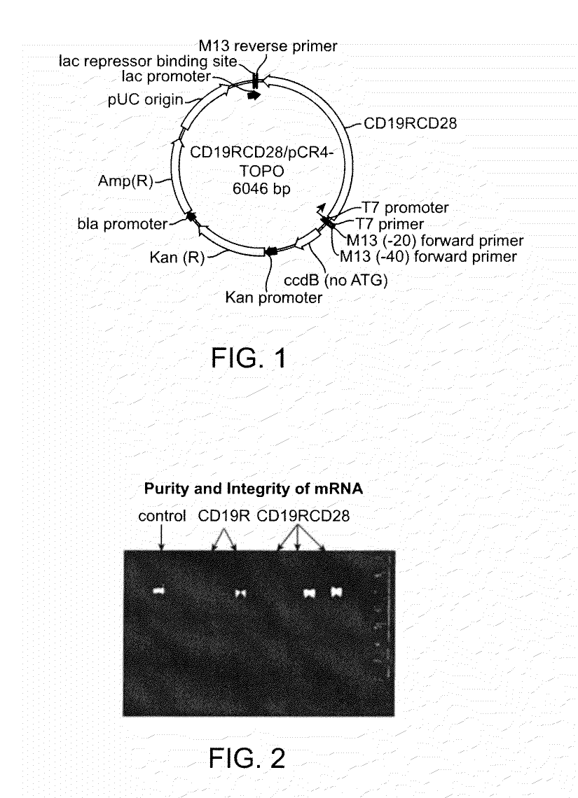

In this example, CD19-specific chimeric antigen receptor (CAR) was used as the transgene to be expressed in T cells. To evaluate the electroporation of desired mRNA, and whether electroporated mRNA can be expressed in primary cells and in cell lines, a T7 promoter was generated based on vectors containing second generation CAR designated CD19RCD28 (FIG. 1). Integrity of these vectors was determined by standard molecular biology methods. To generate mRNA specific for CD19R and CD19RCD28 from this vector, the DNA vectors were linearized (FIG. 2A) and the mRNAs were prepared using an MEGAscript kit (Ambion, Tex.) according to manufacturer instructions. Purity and integrity of mRNAs were determined by gel electrophoresis (FIG. 2B). Purified RNAs were then electroporated into a Jurkat T-cell line, a NK92 cell line and primary NK cells using Amaxa Biosystems Nucleofector™ II and the expression of CD19R and CD19RCD28 were determined by FACS analysis (FIG. 3A).

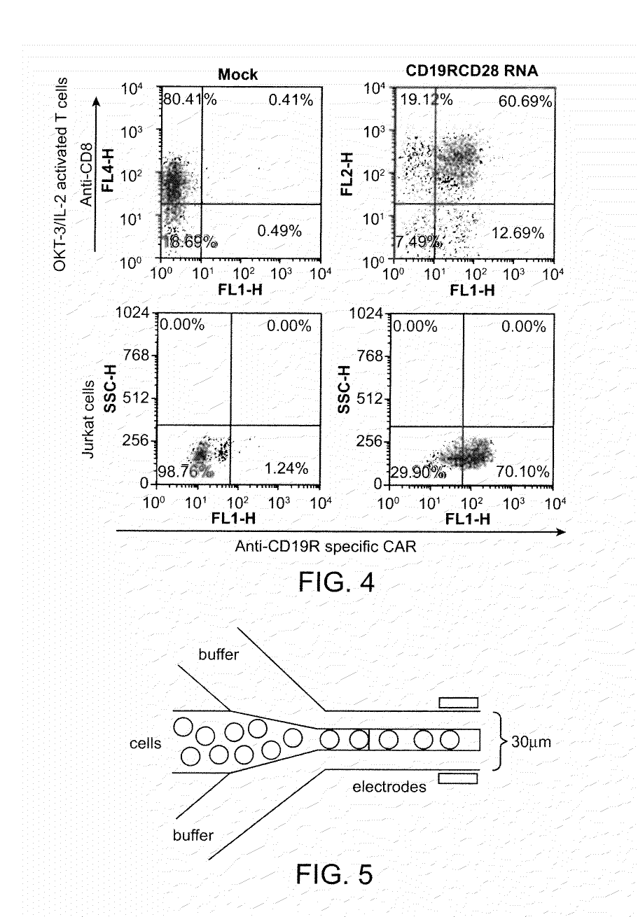

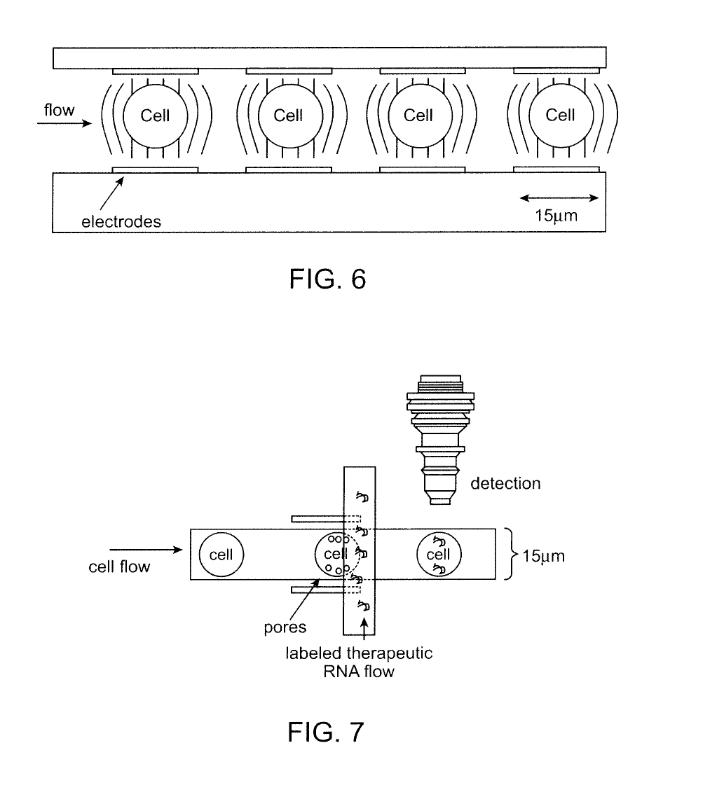

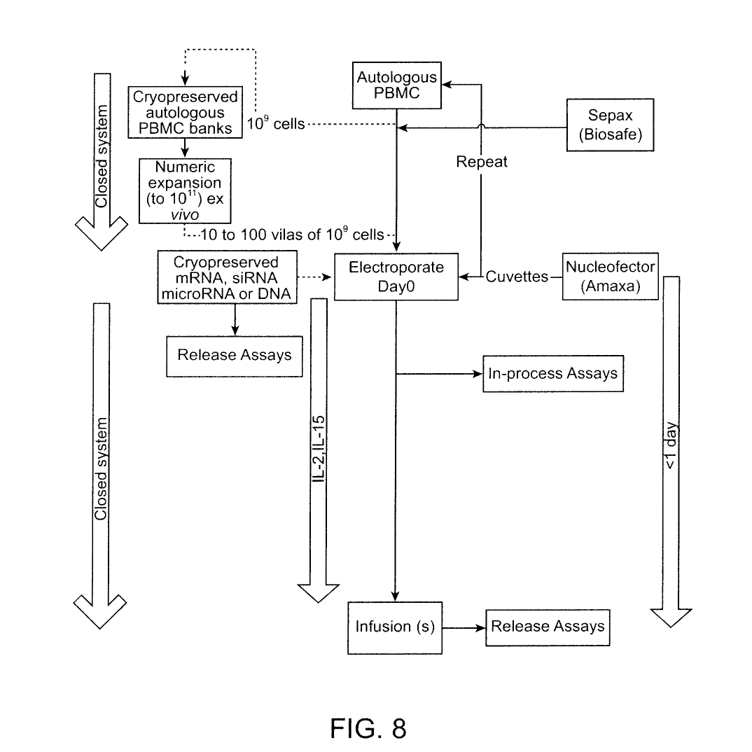

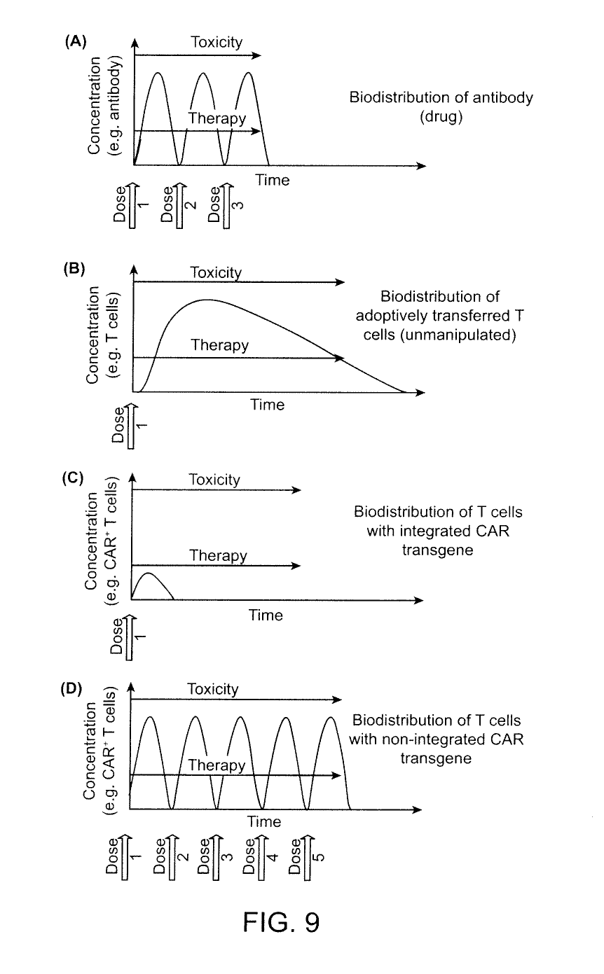

As seen in FIG. 3A, when the NK 92 cell line was electroporated with CD19RCD28, 20% of the cells were positive for 2D3-Alexa labeled CD19. In contrast however, the primary NK cells were negative for CD19R. These data demonstrate that electroporation conditions for primary NK cells would be different then NK cell lines. RNA electroporation in the Jurkat T-cell line was also successful, with 10% of the cells positive for CD19R. When Cy5 labeled CD19R was electroporated into the cells and FACS analysis performed to determine the presence of mRNA (FIG. 3B), the labeled mRNA could be detected in NK92 and Jurkat cells for up to 24 hrs (FIG. 4).

### Example 2

### Non-Integrated Plasmid (NIP) Study

Anti-CD20-IL-2 ICK was demonstrated to bind specifically to CD20⁺ tumors as well as IL-2R⁺ T cells and infusing a combination of anti-CD20-IL-2 ICK with CD19R⁺ T cells improves in vivo T-cell persistence leading to an augmented clearance of CD20⁺CD19⁺ tumor, beyond that achieved by delivery of the ICK or T cells alone.

### Plasmid Expression Vectors

The plasmid vector CD19R/ffLucHyTK-pMG co-expresses the CD19R chimeric immunoreceptor gene and the tripartite fusion gene ffLacHyTK (22). Truncated CD19, lacking the cytoplasmic domain (Mahmoud M S, et al., Blood (1999) 94:3551-8), was expressed in ffLucHyTK-pMG to generate the plasmid tCD19/ffLucHyTK-pMG to co-express the CD19 and ffLucHyTKtransgenes. Bifunctional hRLucZeo fusion gene that co-expresses Renilla koellikeri (Sea Pansy) luciferase hRLuc and zeomycin-resistance gene (Zeo) was cloned from the plasmid pMOD-LucSh (InvivoGen, San Diego, Calif.) into peDNA3.1⁺ (lnvitrogen, Carlsbad, Calif.), to create the plasmid hRLuc:Zeocin-pcDNA3.1. Propagation of cell Lines and primary human T cells

Daudi, ARH-77, Raji, SUP-B15, K562, cells were obtained from ATCC (Manassas, Va.) and Granta-519 cells from DSMZ (Braunschweig, Germany). An EBV-transformed lymphoblastoid cell line (LCL) was kindly provided by Drs. Phillip Greenberg and Stanley Riddell (Fred Hutchinson Cancer Research Center, Seattle, Wash.). These cells were maintained in tissue culture as described (Serrano L M, et al., Blood (2006) 107:2643-52). IL-2Rβ⁺ TF-Iβ cells were kindly provided by Dr. Paul M. Sondel, (University of Wisconsin, Madison, Wis.) (Farner N L, et al., Blood (1995) 86:4568-78). Human T-cell lines were derived from UCB mononuclear cells after informed consent and cultured as previously described (Cooper L J, et al., Blood (2003) 101:1637-44; Riddell S R, Greenberg P D, J Immunol Methods 1990;128:189-201).

### Immunocytokines (ICKs)

The anti-CD20-IL-2 (DI-Leu16-IL-2) ICK was derived from a de-immunized anti-CD20 murine mAb (Leul6). Anti-GD₂-IL-2 (14.18-IL-2) which recognizes GD₂ disialoganglioside served as a control ICK with irrelevant specificity for a B-lineage tumor line used in this study (EMD Lexigen Research Center, Billerica Mass.) (Gillies S D, et al., Proc Nad Acad Sci U S A 1992;89:1428-32).

### Non-Viral Gene Transfer of DNA Plasmid Vectors

OKT3-activated UCB-derived T cells were genetically modified by electroporation with CD19R/ffLucHyTK-pMG (Serrano L M, et al., Blood (2006) 107:2643-52). ARH-77 was electroporated with hRLuc:Zeocin-pcDNA3.1 using the Multiporator device (250V/40 μsec, Eppendorf, Hamburg, Germany) and propagated in cytocidal concentration (0.2 mg/mL) of zeocin (InvivoGen).

### Flow Cytometry

Fluorescein isothiocyanate (FITC), or phycoerythrin (PE), conjugated reagents were obtained from BD Biosciences (San Jose, Calif.): anti-TCRαβ, anti-CD3, anti-CD4, anti-CD8, anti-CD25, and anti-CD122. F(ab′)₂ fragment of FITC-conjugated goat anti-human Fcy, (Jackson Immunoresearch, West Grove, Pa.) was used at 1/20 dilution to detect cell-surface expression of CD19R transgene. Leul6 and anti-CD20-IL-2 ICK (100 μg each) were conjugated to Alexa Fluor 647 (Molecular Probes, Eugene Oreg.). Data acquisition was on a FACS Calibur (BD Biosciences) using CellQuest version 3.3 (BD Biosciences) and analysis was undertaken using FCS Express version 3.00.007 (Thornhill, Ontario, Canada).

### Chromium Release Assay

The cytolytic activity of T-cells was determined by 4-hour chromium release assay (CRA). CD19 specific T cells were incubated with 5×10³ chromium labeled target cells in a V-bottom 96-well plate (Costar, Cambridge, Mass.). The percentage of specific cytolysis was calculated from the release of ⁵¹Cr using a TopCount NXT (PerkinElmer Life and Analytical Sciences, Inc, Boston, Mass.). Data are reported as mean±SD.

### Immunofluorescence Microscopy

CD19R⁺ T cells (10⁶) and CD19⁺CD20⁺tumor cells (10⁶) were centrifuged at 200 g for 1 min and incubated at 37° C. for 30 minutes. After gentle re-suspension, the cells were sedimented, supernatant was removed, and the pellet was fixed for 20 min with 3% parafomaldehyde in PBS on ice. After washing, the fixed T cell-tumor cell conjugates were incubated for 30 minutes at 4° C. with anti-CD3-FITC or Alexa Fluor 647-conjugated anti-CD20-IL-2 ICK. Nuclei were counterstained with Hoechst 33342 (Molecular Probes. Eugene, Oreg.) (0.1 μg/mL). Cells were examined on a Zeiss LSM 510 META NLO Axiovert 200M inverted microscope. Hoechst 33342 was excited at 750 nm using Coherent Ti:Sapphire multiphoton laser, Alexa Fluor 647 at 633 nm using Helium-Neon laser, and FITC at 488 nm using Argon ion laser. Images were acquired with a Zeiss plan-neofluar 20X/0.5 air lens or plan neofluar 40X/1.3 NA oil immersion lens and fields of view were then examined using Zeiss LSM Image Browser Version 3,5,0,223 (configuration at www.cityofhope.org/LMC/LSMmett.asp).

### Persistence of Adoptively Transferred T Cells

Prior to the initiation of the experiment, 6-10 week old female NOD/scid (NOD/LtSz-Prkdcscid/J) mice (Jackson Laboratory, Bar Harbor, Me.) were γ-irradiated to 2.5 Gy using an external ¹³⁷Cs-source (J L Shepherd Mark I Irradiator, San Fernando, Calif.) and maintained under pathogen-free conditions at COH Animal Resources Center. On day-7 the mice were injected in the peritoneum with 2×10⁶ hRLuc⁺ CD19⁺CD20⁺ARH-77 cells. Tumor engraftment was evaluated by biophotonic imaging and mice with progressively growing tumors were segregated into four treatment groups to receive 10⁷ CD19-specific T-cells (day 0) either alone or in combination with 75,000 U/injection (equivalent to ˜25 μg ICK(25)) IL-2 (Chiron, Emeryville, Calif.), 5 μg/injection anti-CD20-IL-2 ICK (DI-Leu16-IL-2) or 5 μg/injection anti-GD₂-IL-2 ICK, given by additional separate intraperitoneal injections. Animal experiments were approved by COH institutional committees.

In vivo Efficacy of Combination Immunotherapies

Six to ten week old γ-irradiated NOD/scid mice were injected with 2×10⁶ hRLuc⁺ CD19⁺CD20⁺ARH-77 cells in the peritoneum. Sustained tumor engraftment was documented within 7 days of injection by biophotonic imaging. Mice in the four treatment groups received combinations of CD19-specific T cells (10⁷ cells in the peritoneum on day 0), anti-CD20-IL-2 ICK or anti-GD₂-IL-2 ICK (5 μg/injection in the peritoneum).

### Biophotonic Imaging

Anaesthetized mice were imaged using a Xenogen IVIS 100 series system as previously described (Cooper L J, et al., Blood (2005) 105:16221-31). Briefly, each animal was serially imaged in an anterior-posterior orientation at the same relative time point after 100 μL (0.068 mg/mouse) of freshly diluted Enduren™ Live Cell Substrate (Promega, Madison, Wis.), or 150 μL (4.29 mg/mouse) of freshly thawed D-luciferin potassium salt (Xenogen, Alameda, Calif.) solution injection. Photons were quantified using the software program “Living Image” (Xenogen). Statistical analysis of the photon flux at the end of the experiment was accomplished by comparing area under the curve using two-sided Wilcoxon rank sum test. Biologic T-cell half life was calculated as A=I×(½)^{(t/h)} (A=flux at time t, I=day 0 flux, h=rate of decay).

### Redirecting T Cells Specificity for CD19

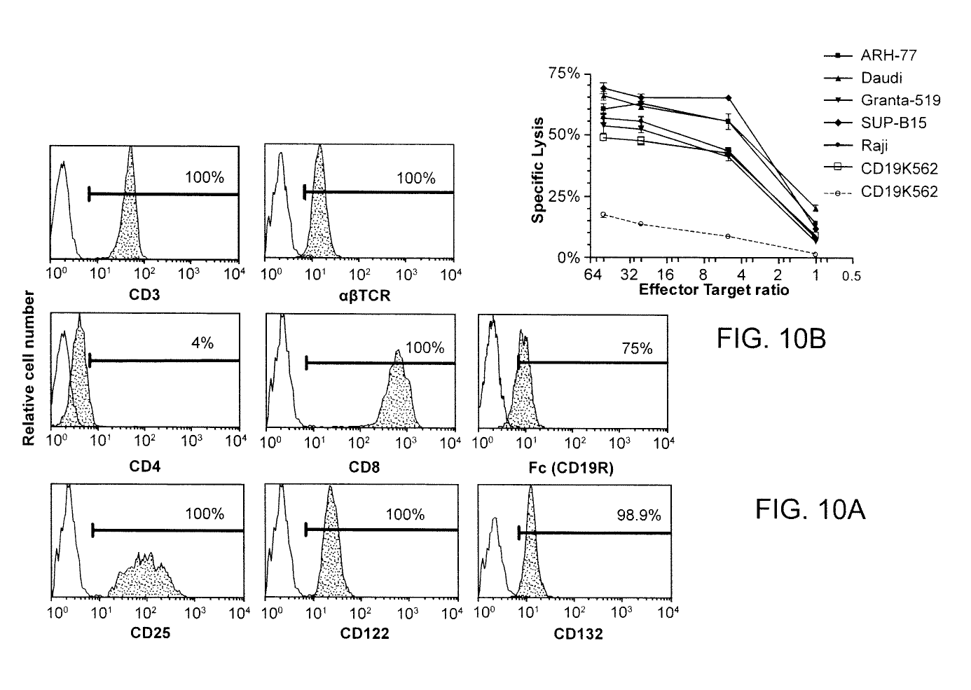

The genetic modification of UCB-derived T cells to render them specific for CD19 was accomplished by non-viral electrotransfer of a DNA expression plasmid designated CD 19R/ffLucHyTK-pMG, that codes for the CD19R transgene (Cooper L J, et al., Blood (2003) 101:1637-44) and a recombinant multi-function fusion gene that combines firefly luciferase (ffLuc), hygromycin phosphotransferase and herpes virus thymidine kinase (HyTK) (Lupton S D, et al., Mol. Cell Biol. (1991) 11:3374-8), permitting in vitro selection of CD19R⁺ T cells with cytocidal concentration of hygromycin B and in vivo imaging after infusion of D-luciferin. Genetically modified ex vivo expanded T cells were CD8⁺; expressed components of the high-affinity IL-2 receptor (IL-2R) and CD19R transgene, as detected using a Fc-specific antibody (FIG. 10A). CD19R⁺ T cells could specifically lyse leukemia and lymphoma targets expressing CD19 with ˜50-70% of CD19⁺ tumor cells killed at an E:T ratio of 50:1 in a 4 hour CRA (FIG. 10B). The variability of lysis of the various B-cell lines could be attributed to the expression of various cell surface markers particularly the adhesion molecules (Cooper L J, et al., Blood (2003) 101:1637-44). Specific lysis of CD19⁺ K562 compared to CD19^{neg} K562 cells demonstrated that the killing of CD19⁺ tumor targets occurred through the chimeric immunoreceptor.

### Binding of Anti-CD20-IL-2 ICK

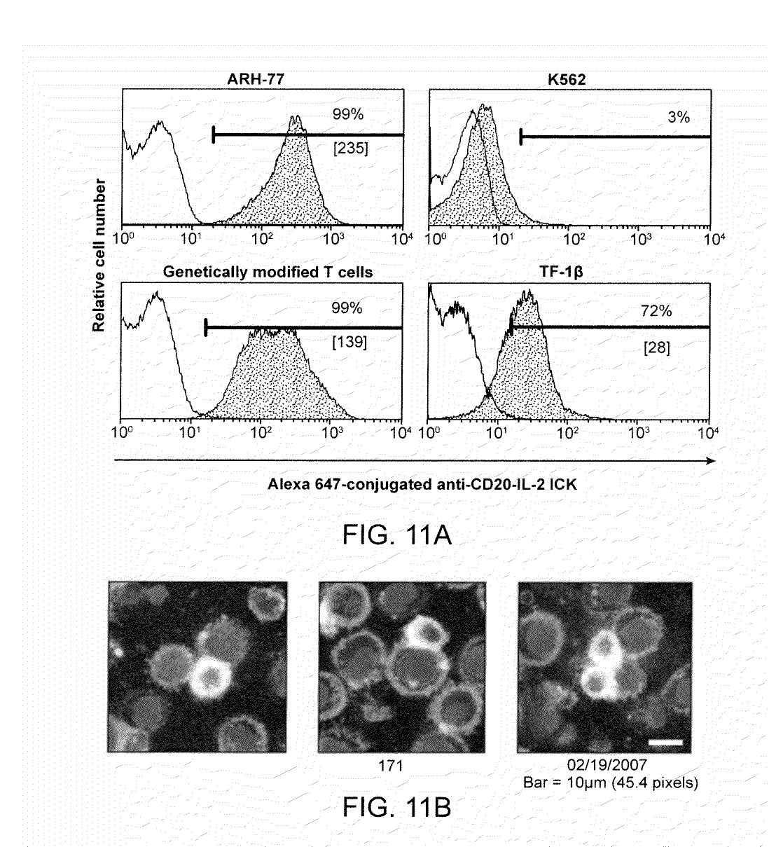

The ability of the anti-CD20-IL-2 ICK to bind to both B-lineage tumors and T cells was examined using flow cytometry and confocal microscopy. This ICK bound to CD20⁺ ARH-77 but not CD20^{neg} SUP-B15 and K562 cells, consistent with recognition of parental Leu16 mAb for CD20 (FIG. 11A) (Rentsch B., et al., Eur. J. Haematol. (1991) 47:204-12). The anti-CD20-IL-2 ICK, but not parental Leul6 mAb, bound to CD25⁺ genetically modified T cells and to TF-1β, a tumor cell line genetically modified to express CD122 (IL-2Rβ) (Farner N L, et al., Blood (1995) 86:4568-78), which is consistent with binding of chimeric IL-2 via the IL-2R (FIG. 11A). The greater median fluorescent intensity (MFI) on T cells, compared with TF-1β, is consistent with binding of the ICK to the high-affinity IL-2R. Immunofluorescence confocal microscopy was performed to evaluate the localization of ICK on conjugates of CD19-specific T cells and CD20⁺ tumors. The confocal micrographs demonstrated cell-surface labeling of conjugates of tumor and T cells with Alexa Fluor 647-conjugated anti-CD20-IL-2 ICK (red) and T cells labeled with FITC-conjugated anti-CD3 (green). Areas of overlapping binding between deposition of ICK and anti-CD3 is depicted by a yellow color (FIG. 11B). These results show that T cells exhibit co-localization of CD3 and ICK on their surface initially but as they form a synapse with the tumor cell there seems to be a rearrangement of IL2R on the T cells towards the synapse leading to the presence of yellow signal extending well outside the synapse and leaving a green pocket opposite the synapse. The Alexa Fluor 647-conjugated parental anti-CD20 Leu16 mAb, lacking the chimeric IL-2 domain, binds CD20⁺ tumors, but not the genetically modified T cells (data not shown). In aggregate these data show that anti-CD20-IL-2 ICK can bind to CD20 molecules on B-lineage tumors and IL-2R on T cells and furthermore that this ICK can be deposited at the interface between tumor and T cells.

In vivo T-Cell Persistence given in Combination with ICK

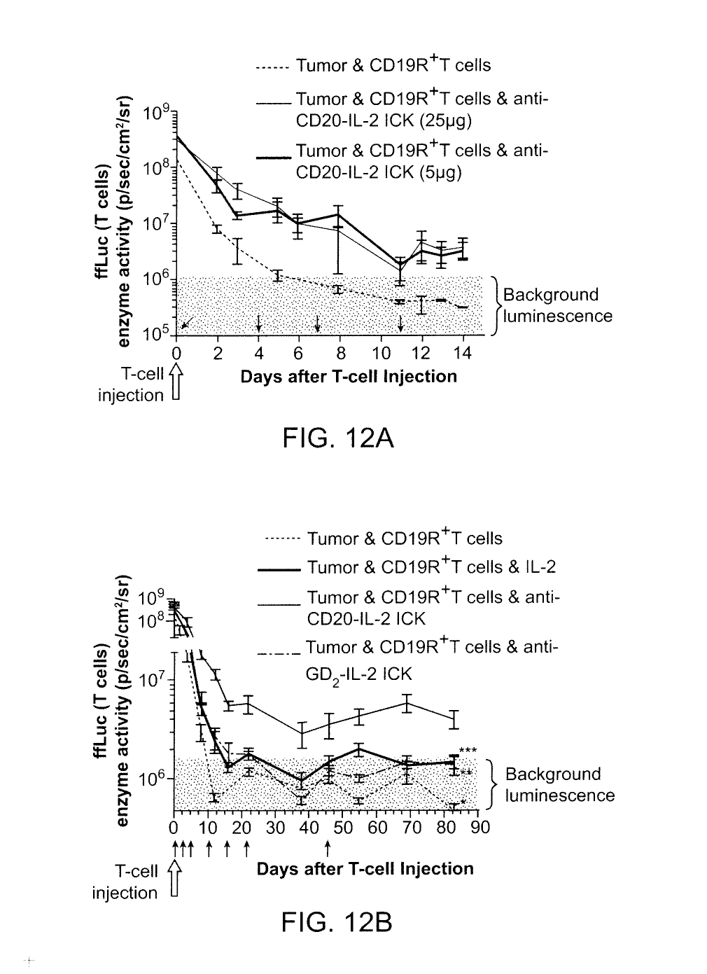

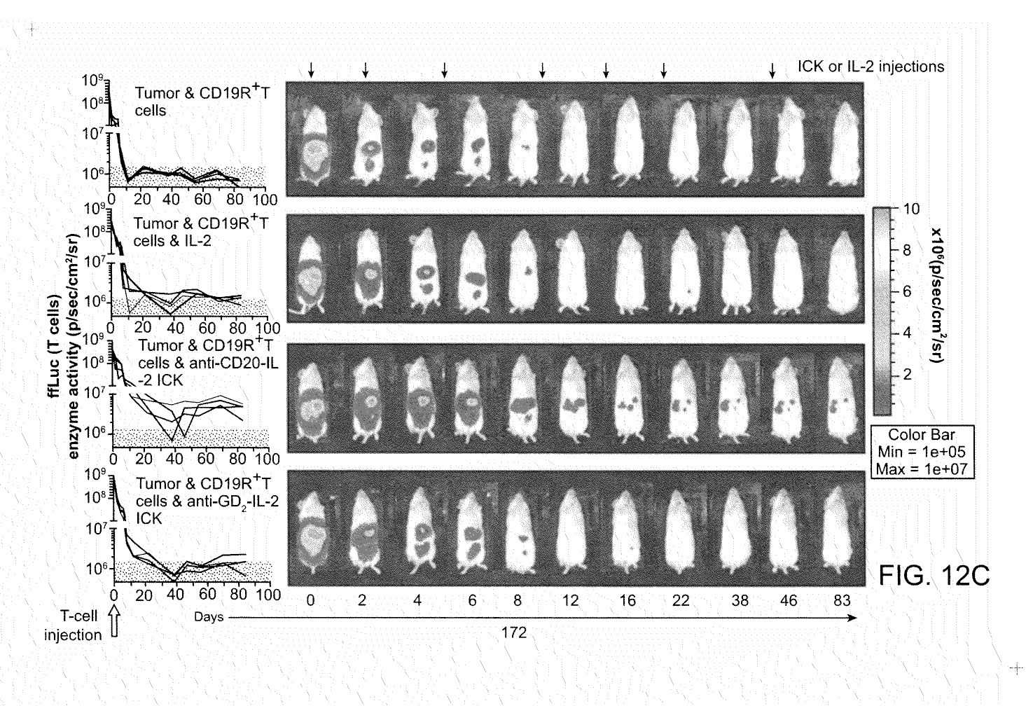

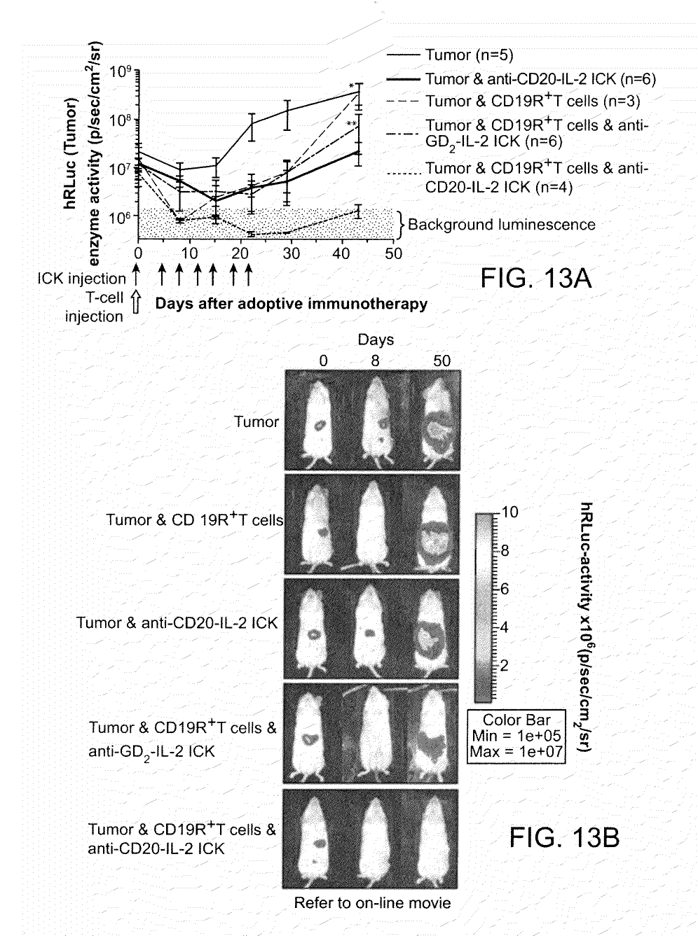

Having determined that the anti-CD20-IL-2 ICK could bind to tumor and T cells, whether infusions of anti-CD20-IL-2 ICK can improve the in vivo persistence of adoptively transferred genetically modified CD8⁺ T cells was evaluated. To achieve sustained loco-regional depositions of the anti-CD20-IL-2 ICK, the tumor line ARH-77 was chosen as a target for immunotherapy, since this is relatively resistant to killing by anti-CD20-specific mAb (Treon S P, et al., J. Immunother. (2001) 24:26371), and these results were confirmed in vivo in NOD/scid mice using rituximab. Initially, a dose of ICK was established that could both improve the in vivo survival of CD8⁺CD19R⁺ffLuc⁺ T cells, compared with adoptive immunotherapy in the absence of ICK, and not statistically alter tumor growth as monotherapy (FIG. 13). It was demonstrated that an ICK dose of both 5 and 25 μg can improve the persistence of infused T cells resulting in a T-cell ffLuc-derived signal detectable above background luminescence measurements (≦10⁶ p/sec/cm²/sr) 14 days after adoptive immunotherapy (FIG. 12A). Biologic half life of the infused T cells was determined by calculating the rate of T-cell decay (ftLuc activity) at the end of the experiment and expressed as the number of days required by the cells to achieve half the initial (Day 0) flux. Indeed, the biological half-life of the infused T cells was twice as long in mice that received ICK (1.09 d) compared with T cells given alone (0.43 d). As a further indication that infusion of the ICK may enhance the survival of adoptively transferred T cells, an approximately 300% (3-fold) increase was observed in the ffLuc-derived signal (day 12) as compared to day 11 when the ICK was injected in both the groups. As the relative in vivo T-cell persistence was similar for both of the ICK doses (p=0.86), 5 μg per ICK injection was used for subsequent experiments, a dose equivalent to ˜15,000 units of human recombinant IL-2 (Gillies S D, et al., Blood 2005;105:3972-8).

To determine if the improved T-cell persistence was due to the binding of the ICK in the ARH-77 tumor microenvironment, a control ICK (anti-GD₂-IL-2 ICK) which does not bind to GD₂^{neg} ARH-77 was used. Furthermore, the ability of the anti-CD20-IL-2 ICK to potentiate T-cell survival was compared with administration of exogenous recombinant human IL-2. Longitudinal measurement of ffLuc-derived flux revealed that the infused T cells persisted longer in mice that received anti-CD20-IL-2 ICK, as compared to the untreated (p=0.01), IL-2-treated (p=0.02) and control ICK-treated (p=0.05) groups (FIG. 12B, 12C); the biological half lives of T cells in the groups being 1.7, 0.5, 1.0 and 0.7 days respectively. There was a difference (p<0.05) in the in vivo persistence of T cells accompanied by IL-2, compared with T cells given without this cytokine, which is consistent with the dependence of these T cells to receive T-cell help in the form of exogenous IL-2 to survive in vivo. No apparent difference was observed in the persistence (p=0.5) or biologic half-life (p=0.2) of adoptively transferred T cells between the mice receiving exogenous IL-2 or control ICK. These data support the hypothesis that the loco-regional deposition of the anti-CD20-IL-2 ICK at the CD19⁺CD20⁺ tumor site significantly augments in vivo persistence of CD8⁺ CD 19-specificT cells.

In vivo Efficacy of ICK in Combination with CD19-Specific T Cell to Treat Established B-Lineage Tumor

In vivo investigation was performed to determine whether the ICK-mediated improved persistence of genetically modified CD19-specific T cells could lead to augmented clearance of established CD19⁺CD20⁺ tumor. A dose of T cells (10⁷ cells) was selected since this dose by itself does not control long-term tumor growth (FIG. 13). CD19-specific CD8⁺ T cells were adoptively transferred into groups of mice bearing established CD19⁺CD20⁺hRLuc⁺ ARH-77 tumor along with anti-CD20-IL-2 ICK, or control anti-GD₂-IL-2 ICK. Tumor growth was serially monitored by in vivo bioluminescent imaging (BLI) of ARH-77 tumor-derived hRLuc enzyme activity. Mice that received both CD19-specific T cells and anti-CD20-IL-2 ICK experienced a reduction in tumor growth with 75% of mice obtaining complete remission, as measured by BLI, at the end of the experiment (50 days after adoptive immunotherapy) (FIG. 13). It was found that the combination therapy of CD19R⁺ T cells and anti-CD20-IL-2 ICK was effective in reducing tumor growth as compared to no immunotherapy (p=0.01) and T cells given with an equivalent dosing of the control ICK (p=0.03). Even though the tumor burden seems to be increasing in the treated group, no visible tumor as seen by hRLuc signal was observed at the end of the experiment, as the flux remained below background level, consistent with a complete anti-tumor response. Mouse groups receiving T cells alone or T cells with control ICK showed a similar pattern of tumor growth, with an initial reduction around day 8, followed by relapse. All mice in the control group, which received no immunotherapy, experienced sustained tumor growth. Similar tumor growth kinetics were observed in mice that did or did not receive anti-CD20-IL-2 ICK in the absence of T cells (p>0.05 through day 50) and this is presumably a reflection of the dose regimen chosen for the ICK in this experiment. Increased doses of T cells or anti-CD20-IL-2 ICK delivered as monotherapies results in a sustained anti-tumor effect, but using these doses would preclude the ability to measure the ability of the ICK to potentiate T-cell persistence and improve tumor killing.

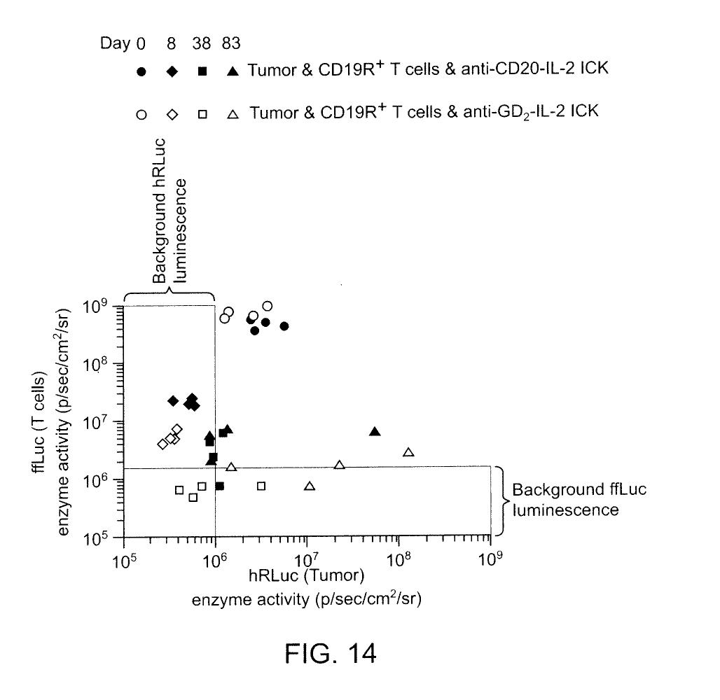

The ability to measure both ffLuc and hRLuc enzyme activities in the same mice allowed the determination of whether the persistence of adoptively transferred T cells directly correlated with tumor size for individual mice. This was accomplished by plotting ffLuc-derived T-cell flux versus hRLuc-derived tumor-cell flux from FIG. 12. Both groups of mice, which received CD19-specific T cells along with anti-CD20-IL-2 ICK/anti-GD2-IL-2 ICK, showed a drop in tumor burden at day 8, which is due to the T cells infused. However, the highest numbers of T cells (ffLuc activity; mean flux 4.7×10⁶ vs 1.5×10⁶ p/sec/cm²/sr) and lowest tumor burden (hRLuc activity; mean flux 1.4×10⁷ vs 4×10⁷ p/sec/cm²/sr) by day 83 (FIG. 14) was observed in the group receiving CD20-ICK, when compared to the control ICK-treated group. This analysis demonstrates that half the mice achieve an anti-tumor response (absence of detectable hRLuc activity) after combination immunotherapy with CD19R⁺ T cells and anti-CD20-IL-2 ICK. It was noted that there was continued T-cell persistence (ffLuc activity) in the anti-CD20-IL-2 ICK-treated group as compared to the control ICK treated group (p<0.05) at day 83. Although tumor burden (hRLuc activity) was reduced in the CD20-ICK as compared to the control ICK treated group at day 83, no statistical significance was observed. Thus, a trend towards continued T-cell persistence and desired anti-tumor effect in the CD20-ICK treated group was noted.

The above results demonstrate, for the first time, that BLI can be used to connect the persistence of T cells to an anti-tumor effect. These data further reveal that the mice which receive the tumor-specific immunocytokine control their tumor burden to a greater extent than the mice which receive the control immunocytokine (which does not bind the tumor). As a treatment for minimal residual disease in patients undergoing bone marrow transplantation this combination therapy demonstrates the ability to keep the disease relapse in check for almost 3 months in this mouse model.

In aggregate, these data demonstrate that the combination of anti-CD20-IL-2 ICK and CD19R⁺ T cells results in augmented control of tumor growth, as is predicted from the in vivo T-cell persistence data.

It was demonstrated that anti-CD20-IL-2 ICK specifically binds to CD20⁺ tumor, that infusions of the anti-CD204L-2 ICK can augment persistence of adoptively transferred CD19-specific T cells in vivo, and that this leads to improved control of an established CD19⁺CD20⁺ tumor. These observations can be due to the deposition of IL-2 at sites of CD20 binding which provides a positive survival stimulus to infused CD19R⁺IL-2R⁺ effector T cells residing in the tumor microenvironment.

The development of an anti-CD20-IL-2 ICK has implications for future immunotherapy of B-lineage malignancies. For while Rituximab has been extensively used to treat CD20⁺ malignancies (Foran J M, J. Clin. Oncol. (2000) 18:317-24; Maloney D G, et al., Blood 1997;90:2188-95; Reff M E, et al., Blood (1994) 83:435-45), some patients become unresponsive to this mAb therapy leading to disease progression (McLaughlin P, et al., J. Clin. Oncol. (1998) 16:2825-33). The development of an anti-CD20-IL-2 ICK with its ability to activate immune effector cells, may rescue these patients. Modifications other than the addition of cytokines (Lode H N, Reisfeld R A., Immunol. Res. (2000) 21:279-88; Penichet M L, Morrison S L, J. Immunol. Methods (2001) 248:91-101), such as radionucleotides (Jurcic J G, Scheinberg D A, Curr. Opin. Immunol. (01994) 6:715-21), and cytotoxic agents (Kreitman R J, et al., J. Clin. Oncol. (2000) 18:1622-36; Pastan I., Biochim. Biophys. Acta (1997) 1333:1-6), may also improve the therapeutic potential of unconjugated clinical-grade mAbs. Indeed combining mAb-therapy with therapeutic modalities that exhibit non-overlapping toxicity profiles is an attractive strategy to improving the anti-tumor effect without compromising patient safety.

The combination therapy for treating B-lineage tumors described herein combines ICK with T-cell therapy. The two immunotherapies used, anti-CD20-IL-2 ICK and CD19-specific T cells, have the potential to improve the eradication of tumor since (i) the targeting of different cell-surface molecules reduces the possibility emergence of antigen-escape variants, (ii) the mAb conjugated to IL-2 can recruit and activate effector cells (such as CD19-specific T cells) expressing the cytokine receptor in the tumor microenvironment, and (iii) T cells can kill independent of host factors which may limit the effectiveness of mAb-mediated complement dependent cytoxicity (CDC) and antibody dependent cell cytotoxicity (ADCC) (12-15). These immunotherapies will target both malignant and normal B cells. However, as loss of normal B-cell function has not been an impediment to Rituximab therapy and as clinical conditions associated with hypogammaglobulinemia could be corrected with infusions of exogenous immunoglobulin, a loss of B-cell function may be an acceptable side-effect in patients with advanced B-cell leukemias and lymphomas receiving CD19- and/or CD20-directed therapies.

Another advantage of ICK-therapy is that the loco-regional delivery of T-cell help in the form of IL-2, may avoid the systemic toxicities observed with intravenous infusion of the IL-2 cytokine (43-45) and this may be particularly beneficial in the context of allogeneic hematopoietic stem-cell transplant (HSCT). It has been reported that UCB-derived CD8⁺ T cells can be rendered specific for CD19 to augment the graft-versus-tumor effect after HSCT and since the ICK improves the in vivo immunobiology of UCB-derived CD19-specific T cells, combining the two immunotherapies after UCB transplantation may be beneficial.

Alternative ICK's and T cells with shared specificities for tumor types other than B-lineage malignancies could also be considered for combination immunotherapy. For example, ICK's might be combined with T cells which have been rendered specific by the introduction of chimeric immunoreceptors for breast (46;47), ovarian (48), colon (49), and brain (50) malignancies. Furthermore, ICK's bearing other cytokines might be infused with T cells to deliver IL-7, IL-15, or IL-21 to further augment T-cell function in the tumor microenvironment.

Currently, the lineage-specific cell-surface molecules CD19 and CD20 present on many B-cell malignancies are targets for both antibody- and cell-based therapies. Coupling these two treatment modalities is predicted to improve the anti-tumor effect, particularly for tumors resistant to single-agent biotherapies. This can be demonstrated using an immunocytokine (ICK), composed of a CD20-specific monoclonal antibody (mAb) fused to biologically-active IL-2, combined with ex vivo-expanded human umbilical cord blood(UCB)-derived CD8⁺ T cells, that have been genetically modified to be CD19-specific, for adoptive transfer after allogeneic hematopoietic stem-cell transplant. It was shown that a benefit of targeted delivery of recombinant IL-2 by the ICK to the CD19⁺CD20⁺ tumor microenvironment is improved in vivo persistence of the CD19-specific T cells and this results in an augmented cell-mediated anti-tumor effect.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed:

1. A device for high throughput transfection of living cells, comprising:
optionally, a cell selection component capable of being operatively coupled to a source of living cells;
optionally, a cell focusing component:
capable of being operatively coupled to a source of living cells if the cell selection device is not opted for or
operatively coupled to the cell selection component;
optionally, a cell activation component:
capable of being operatively coupled to a source of living cells if both the cell selection and cell focusing components are not opted for or, if the cell selection component is not opted for but the cell focusing component is, operatively coupled to the cell focusing component or if the cell focusing component is not opted for and the cell selection component is, operatively coupled to the cell selection component;
a high throughput electroporation component:
capable of being operatively coupled to a source of living cells or
if opted for, operatively coupled to the cell activation component or
if the cell activation component is not opted for and the cell focusing component is, operatively coupled to the cell focusing component or
if both the cell activation component and the cell focusing component are not opted for and the cell selection component is, operatively coupled to the cell selection component;
a source of DNA and/or RNA operatively coupled to the high throughput electroporation component;
optionally, a transfection detector component operatively coupled to the distal end of the high throughput electroporation component; and,
optionally, a cell separation component operatively coupled to the transfection detector.

2. The device of claim 1, wherein the cell selection component comprises an apheresis component.

3. The device of claim 1, wherein the cell focusing component comprises channels for funneling cells through the electroporation device one cell at a time.

4. The device of claim 1, wherein the cell activation component comprises a chamber having an inlet operatively coupled to a source of activating substance, the chamber also being operatively coupled to the cell selection component, if opted for, the cell focusing component if the cell selection component is not opted for or capable of being coupled to a source of living cells if neither the cell selection nor the cell focusing components are opted for, and an outlet operatively coupled to the electroporation component.

5. The device of claim 1, wherein the high throughput electroporation component comprises a plurality of microfluidic electroporation units, each unit comprising:
a first non-conductive support element, the element having a length with a proximal end and a distal end, a width and a surface;
a first conductive layer disposed over the surface of the first non-conductive support element;
a second non-conductive support element having a length and width substantially the same as the first non-conductive support element and a surface, the second non-conductive support element being substantially parallel to the first non-conductive support element with the surface of the second non-conductive support element facing the surface of the first non-conductive support element;
a second conductive layer disposed over the surface of the second non-conductive support element; wherein:
the first conductive layer is no more than about 100 μm distant from the second conductive layer, the distance being maintained by a plurality of non-conductive spacers; wherein:
the spacers extend from the proximal to the distal ends of the conductive surfaces thereby forming a plurality of channels extending substantially the full length of the conductive surfaces;
a pulse generator in electrical contact with the first conductive layer and the second conductive layer; and,
a positive displacement pump operatively coupled to a proximal end of the plurality of electroporation units; or,
a vacuum pump operatively coupled to a distal end of the plurality of electroporation units.

6. The device of claim 1, wherein the transfection detector component comprises a fluorescence detector.

7. The device of claim 1, wherein the cell separation component comprises channels that separate transfected cells from live-but-not-transfected cells and/for from dead cells.

8. The device of claim 1, wherein all the components are contained in a sealed housing having one or more inlets and one or more outlets for contact with the external environment.

9. The device of claim 8, wherein all the components and the housing are sized to be implantable in the body of a patient.

10. A method of treating a disease, comprising:
identifying a patient afflicted with a disease that is known to be, becomes known to be or is suspected of being responsive to treatment using transfected cells;
providing a source of living cells;
optionally selecting one or more cell types from the living cells;
optionally focusing the source of living cells or the selected cell types;
optionally activating the living cells or the selected cell types;
mixing the living cells or selected cell types with DNA and/or RNA;
electroporating the living cells or selected cell types in the presence of the DNA and/or RNA to give transfected living cells or selected cell types;
optionally detecting cells that have been transfected;
optionally separating transfected cells from living-but-not-transfected cells and/or from dead cells;
administering the transfected cells to the subject, wherein the transfected cells express a therapeutic agent; and,
repeating the above steps until treatment of the patient is complete.

11. The method of claim 10, wherein the living cells or selected cell types are mixed with RNA.

12. The method of claim 10, wherein at no point are the living cells or selected cell types propagated prior to administering them to the patient.

13. The method of claim 11, wherein at no point are the living cells or selected cell types propagated prior to administering them to the patient.

14. The method of claim 10, wherein providing a source of living cells comprises:
providing a sterile container comprising one or more selected cell types;
providing a bodily fluid comprising living cells that has been previously collected from a subject and stored in a sterile container; and,
providing a subject from whom a bodily fluid containing living cells is taken and directly transferred under sterile conditions to the cell selection component, if opted for, the cell activation component, if the cell selection component is not opted for, a cell focusing component, if the cell selection and cell activation components are not opted for or the electroporation component, if the cell selection, cell activation and cell focusing components are not opted for.

15. The method of claim 10, wherein the method is performed recursively.

16. The method of claim 15, wherein performing the method recursively comprises step-wise providing a source of living cells by providing a patient in need of treatment, collection of a bodily fluid from the patient, subjecting the cells to the method of claim 8 and delivering transfected cells back into the patient and repeating the process as necessary, all under sterile conditions.

17. The method of claim 16, wherein transfection is transient.

18. The method of claim 16, wherein performing the method recursively comprises using Nucleofector® to electroporate the cells.

19. The method of claim 15, wherein performing the method recursively comprises continuously collecting the bodily fluid from the patient, continuously subjecting the bodily fluid to the method of claim 8 and continuously delivering the transfected cells back into the patient in a closed, sterile cycle.

20. The method of claim 19, wherein transfection is transient.

21. The method of claim 19, wherein performing the method recursively comprises using the plurality of high throughput microfluidic electroporation units of claim 5.

22. The method of claim 10, wherein taking a bodily fluid from a patient comprises venipuncture, aphersis, an in-dwelling central catheter, a central intravenous catheter or a combination thereof.

23. The method of claim 22, wherein the bodily fluid is blood or a component of blood.

24. The method of claim 10, wherein selecting one or more cell types comprises apheresis.

25. The method of claim 10, wherein the one or more selected cell types are selected from the group consisting of T cells, NK cells, B cells, dendritic (antigen presenting) cells, monocytes, reticulocytes, stem cells, tumor cells umbilical cord blood-derived cells, peripheral-blood derived cells and combinations thereof.

26. The method of claim 25, wherein the stems cells are selected from the group consisting of hematopoitic stem cells and mesenchymal stem cells.

27. The method of claim 25, wherein the one or more selected cell types are selected from the group comprising T cells, NK cells or a combination thereof.

28. The method of claim 27, wherein activating the T cells and/or NK cells comprises contacting the cells with a cytokine or a growth factor.

29. The method of claim 28, wherein the cytokine is IL-2.

30. The method of claim 10, RNA is selected from the group consisting of mRNA, microRNA and siRNA.

31. The method of claim 10, wherein the RNA and/or DNA code for a biotherapeutic agent.

32. The method of claim 31, wherein the biotherapeutic agent is selected from the group consisting of a chimeric antigen receptor, an enzyme, a hormone, an antibody, a clotting factor, a Notch ligand, a recombinant antigen for vaccine, a cytokine, a cytokine receptor, a chemokine, a chemokine receptor, an imaging transgene, a co-stimulatory molecule, a T-cell receptor, FoxP3, a luminescent probe, a fluorescent probe, a reporter probe for positron emission tomography, a KIR deactivator, hemoglobin, an Fc receptor, CD24, BTLA, a transposase, a transposon for Sleeping Beauty, piggyBac and combinations thereof.

33. The method of claim 10, wherein the patient is a mammal.

34. The method of claim 33, wherein the mammal is a human being.

35. The method of claim 34, wherein the human being is a pediatric patient.

36. The method of claim 10, wherein the disease is selected from the group consisting of a pathogenic disorder, cancer, enzyme deficiency, in-born error of metabolism, infection, auto-immune disease, cardiovascular disease, neurological disease, neuromuscular disease, blood disorder, clotting disorder and a cosmetic defect.
